(19) United States
(12) Patent Application Publication
(10) Pub. No.: US 2011/0305748 A1
(43) Pub. Date: Dec. 15, 2011

(54) Vaccines for Pandemic Influenza

(75) Inventors: Christopher H. Clegg, Seattle, WA (US); Steven G. Reed, Bellevue, WA (US); Neal Van Hoeven, Seattle, WA (US)

(73) Assignee: IMMUNE DESIGN, CORP., Seattle, WA (US)

(21) Appl. No.: 13/045,245

(22) Filed: Mar. 10, 2011

Related U.S. Application Data
(60) Provisional application No. 61/313,101, filed on Mar. 11, 2010.

(51) Int. Cl. A61K 39/145; A61P 37/04; A61K 9/127

(52) U.S. Cl. 424/450; 424/210.1; 424/192.1

(57) ABSTRACT

Pharmaceutical and vaccine compositions comprise recombinant hemagglutinin from a pre-pandemic or pandemic influenza virus and an adjuvant comprising GLA. A particularly relevant pre-pandemic influenza virus is H5N1. Kits and methods of using the compositions are also provided.

### RELATED APPLICATIONS

This patent application claims priority to U.S. Provisional Application No. 61/313,101, filed Mar. 11, 2010, which is incorporated by reference in its entirety.

### STATEMENT REGARDING FEDERALLY FUNDED RESEARCH

This invention was made with government support under 5R43A1081383-02, awarded by The National Institute of Allergy and Infectious Diseases. The government may have certain rights in the invention.

### TECHNICAL FIELD

This patent application relates generally to compositions for use as a vaccine for pre-pandemic or pandemic influenza, such as avian flu (e.g., H5N1), swine flu (e.g., H1N1), H7N7, and H9N2. The composition generally comprises a recombinant hemagglutinin from a candidate influenza virus and an adjuvant.

### BACKGROUND

A pandemic is a worldwide epidemic of an infectious disease that takes hold when a new disease emerges, infects humans causing serious illness and spreads readily among people. An influenza pandemic may occur when a new strain or subtype of influenza virus is transmitted from another animal species (e.g. birds or pigs) into a human population lacking immunity from prior exposure to a related virus. Influenza pandemics have been noted as early as the late 1500s and have occurred regularly since then, with the most recent being “Asian Flu” in 1957 (H2N2), “Hong Kong Flu” in 1968 (H3N2) and “Swine Flu” in 2009 (H1N1). The H5N1 “Avian Flu” virus that emerged in the 1990s has infected humans but has not so far caused a pandemic due to inefficient human to human transmission. As global travel and urbanization increases, the spread of influenza epidemics caused by a new virus are predicted to become pandemic very quickly (WHO, “Pandemic preparedness”).

Avian influenza is caused by an Orthomyxovirus that contains a segmented RNA genome. Infection is initiated by binding of the viral hemagglutinin (HA) protein to sialic acid-linked glycoproteins on host cells. The hemagglutinin protein can be divided into 16 subtypes based on antigenic and sequence characteristics. In addition to HA, influenza viruses contain an additional surface protein, neuraminidase (NA) that is involved in release of virus from cells following infection. Neuraminidase proteins are currently divided into 9 subtypes. All possible combinations of the 16 HA and 9 NA subtypes are thought to exist in nature, where wild birds (e.g. ducks) serve as an asymptomatic virus reservoir. Occasionally, influenza viruses are transmitted from wild birds into domestic poultry, where two forms of the disease have been described. One form is common but mild, the other is rare but highly lethal. Highly pathogenic avian influenza virus (HPAI) infections in poultry are commonly caused by the H5 and H7 subtypes. The HA proteins of HPAI viruses are distinguished from other less pathogenic H5 and H7 subtype HA proteins by a set of basic amino acids in the HA cleavage site.

Consistent infection of poultry by certain HPAI viruses, and the relative proximity of poultry and humans has resulted in transmission of four subtypes of avian influenza (H5N1, H7N3, H7N7, and H9N2) into humans. Infection of humans with H7 or H9 subtype viruses generally results in a mild, non-lethal disease. In contrast, infection of humans with H5 subtype viruses results in a severe, often lethal disease (“Cumulative number of Confirmed Human Cases of Avian Influenza A/(H5N1) Reported to WHO, 17 February 2010, www.who.int).

The continued infection of humans by H5N1 viruses, coupled with the geographic expansion of the virus in wild and domestic birds has resulted in a genetic evolution of the H5N1 viruses, which can now be categorized genetically and antigenically into distinct clades and subclades (WHO Global Influenza Program Surveillance Network, Emerging Infectious Dis 11:1515-1521, 2005). Should any of these novel viruses acquire the ability to transmit readily among humans, the likelihood of an H5N1 influenza pandemic is high. Given the high mortality rate associated with H5N1 infection, the potential global impact associated with such a pandemic could be quite severe. As a result, the WHO has initiated the process of a pandemic alert.

The development of vaccines to combat influenza pandemics is a cornerstone of influenza pandemic preparedness plans for WHO and many governments. Vast numbers of safe and effective doses of a pandemic vaccine are needed to meet potential demand in the United States and in the rest of the world. The stockpiling of vaccines against currently circulating pre-pandemic strains is a critical component of current pandemic prevention strategy. It is expected that these vaccines, which will likely not be identical to the newly emerging pandemic virus, will provide sufficient protection while a more specific, strain-matched vaccine is being produced. To date, three H5N1 vaccines (two split-virion and one whole-virion) have received regulatory approval and several additional vaccines are in late stage development.

Many countries are now stockpiling these vaccines and the near-term U.S. goal is to amass enough vaccine to treat 20 million people. However, numerous scientific, technological, and economic challenges complicate preparations for a global flu pandemic. Importantly, the manufacture of pre-pandemic or pandemic vaccines by traditional egg based methods is time consuming, expensive, and will require billions of eggs to manufacture enough vaccine doses to immunize high-risk individuals worldwide. Alternative cell-based strategies to produce attenuated virus are in development, but these reassortant viruses typically contain relatively low levels of vaccine antigen. This low antigen yield is of particular concern in the context of an H5N1 vaccine, as the avian H5 hemagglutinin appears inherently less immunogenic in people than HA from other subtypes. As a result, larger antigen doses are required to induce antibody responses relative to seasonal influenza vaccines. Moreover, given that humans are immunologically naive to the H5 hemagglutinin, a one-dose immunization schedule may not be protective. To illustrate these points, the first FDA-approved vaccine based on H5 clade 1 virus (A/Vietnam/1203/2004) induced “protective” neutralizing titers in just 54% of the study participants and required two doses at 90 μg of HA, 12 times the HA content of the seasonal vaccines (Treanor et al. New Engl J Med 354: 1343-1351, 2006).

Additional technologies are needed that can significantly improve vaccine production capacity, while simultaneously increasing H5N1 immunogenicity. The ideal profile for a pre-pandemic vaccine is that it be easily and inexpensively manufactured and has a long shelf life. Importantly, it should generate robust protective immune responses using minimal antigen, and provide cross-protection against genetically distinct viruses, and ideally, those from different clades. In addition, the vaccine must also be safe.

### SUMMARY

The present invention is directed to compositions for use as vaccines and methods of immunizing subjects with the vaccines, in which the vaccines comprise a recombinant hemagglutinin from a pre-pandemic or pandemic influenza virus and an adjuvant. In one embodiment, the adjuvant is a compound (a DSLP compound) described as a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons. In particular compositions and methods, the adjuvant is GLA (see, e.g., U.S. patent application publication 2008/0131466), which may, in various embodiments, be oil-free, formulated as an oil-in-water emulsion or formulated with other adjuvants such as alum, an aluminum salt. Hemagglutinins of particular interest include H5 from highly pathogenic H5N1 viruses and H1 from H1 N1 (“swine flu”) pandemic influenzas. Compositions may be dosage-sparing and/or the recombinant hemagglutinin may be present in an amount that is dose-sparing. Methods may comprise immunizing subjects with a single injection, i.e., not multiple injections, of the compositions. For example, the composition may be defined by one or more (i.e., any combination of) the following: the rHA is present at an amount that is dose-sparing; the rHA is present at a concentration that does not provide protective immunity in the absence of the adjuvant; the rHA is from a pathogenic strain of avian influenza; the rHA is from a pathogenic strain of H5N1 influenza; the rHA is from clade 1 or clade 2; the rHA is from a pandemic swine flu virus strain; the rHA is from a pandemic H1N1 strain; the composition comprises a single, i.e., not more than one distinct recombinant protein; the amount of rHA per dose is in the range of about 15 to about 0.1 μg; the rHA is expressed from either insect or mammalian cells in order to achieve a preferred level of glycosylation; the rHA is expressed as a fusion protein; the adjuvant is only or includes GLA; the adjuvant is only or includes 3D-MPL, the composition is oil-free; the composition comprises less than about 1% v/v oil or less than about 0.1% v/v oil; the adjuvant is formulated as an aqueous solution prior to being combined with antigen; the adjuvant is formulated in a liposome-containing composition prior to be being combined with antigen; the composition further comprises an aluminum salt or saponin.

In one embodiment, the invention provides a method of immunizing a subject in need thereof against a pre-pandemic or pandemic influenza virus, comprising administering a single injection of a pharmaceutical composition comprising (a) a recombinant hemagglutinin (rHA) from a pre-pandemic or pandemic influenza virus and (b) an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons, where the administration achieves seroconversion after the single injection. In various embodiments, which may individually or in any combination further define the invention: the composition does not include an emulsion; the adjuvant is GLA, the adjuvant is 3D-MPL.

For example, the invention provides, in one embodiment, a pharmaceutical composition comprising: (a) a recombinant hemagglutinin (rHA) from a pre-pandemic or pandemic influenza virus and (b) an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons, for use in a method of immunizing a subject in need thereof against a pre-pandemic or pandemic influenza virus by way of a single injection of the pharmaceutical composition. The pharmaceutical composition may be used in a method of immunizing a population against a pre-pandemic or pandemic influenza virus wherein administration of the composition achieves seroconversion in at least 50%, or at least 60% or more of the population after the single injection. In one aspect, the pharmaceutical composition does not include any oil, i.e., is oil-free, or contains a minimal amount of oil that does not impact the seroconversion efficacy of the compositions, and does not include any oil-containing emulsion. In combination with any of these embodiments, one aspect of the invention is to use GLA as the adjuvant. In another aspect, 3D-MPL may be used as the adjuvant.

As another example, the invention provides a pharmaceutical composition for use in a method of immunizing a subject in need thereof against a pre-pandemic or pandemic influenza virus, where the composition comprisess: (a) a recombinant hemagglutinin (rHA) from a pre-pandemic or pandemic influenza virus and (b) an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons, and wherein the composition is dosage sparing. The pharmaceutical composition for use in a method of immunizing a subject against a pre-pandemic or pandemic influenza virus, wherein the rHA is present at an amount that is dose-sparing. In various embodiments, which may individually or in any combination further define the invention: the rHA is present at a concentration that does not provide protective immunity in the absence of the adjuvant; the composition comprises a single, i.e., not more than one, recombinant protein; the amount of rHA per dose is in the range of about 15 to about 1 μg; and the adjuvant is GLA and the rH5 is from a pathogenic strain of H5N1 influenza.

These and other aspects will become evident upon reference to the following detailed description and attached drawings.

### BRIEF DESCRIPTION OF THE DRAWINGS

FIGS. 1A-C. A single injection of rH5/GLA-SE vaccine protects mice against H5N1 infection. (A) Mice (5/group) were immunized once with 50, 150, 450, 900, or 2700 ng of rH5 (VN) formulated in 2% v/v SE emulsion or GLA-SE adjuvant (20 μg GLA) and then challenged (IN) on day 14 with H5N1 virus (1000×LD₅₀). The mean of the maximum percentage weight loss per group was determined for each group over a two week period. Area under the curves (percent weight loss over time) was determined for each dose of rH5. (B) Percent weight loss in mice at successive days following viral challenge. Mice were vaccinated with 50 ng rH5 formulated with SE alone or with GLA-SE adjuvant, or GLA-SE or SE in the absence of protein. Each data point represents the mean weight loss per group +/− s.e.m. (C) Adjuvant-mediated rH5 protection in c57BI/6 mice. Animals (5/group) were vaccinated once with GLA-SE alone or 50 ng rH5 (VN) formulated with 2% v/v SE alone, 5 μg GLA alone, or 5μg GLA-SE. Mice were challenged (IN) on day 14 with H5N1 Viet Nam 1203 virus (1000×LD₅₀) and monitored for survival and weight loss. Each data point represents the mean weight loss per group +/− s.e.m.

FIG. 2. GLA-SE adjuvant improves survival in vaccinated mice following challenge with a heterologous H5N1 virus. Animals (5/group) were vaccinated with 50 ng homologous (VN) rH5 formulated with GLA-SE adjuvant, 50 ng or 200 ng heterologous (Indo) rH5 formulated in GLA-SE adjuvant, or 200 ng heterologous rH5 alone or rH5 formulated with SE emulsion. Mice were challenged (IN) on day 14 with H5N1 Viet Nam 1203 virus (1000×LD₅₀) and monitored for survival and weight loss. Each data point represents the mean weight loss per group +/− s.e.m.

FIGS. 3A-C. GLA-SE accelerates induction of antigen-specific immunity and recovery from disease. (A) Percent survival in mice as a function of days vaccinated prior to challenge. Mice were immunized with 50 ng rH5 alone or rH5 formulated with SE or GLA-SE, or GLA-SE alone and challenged (IN) on day 0, 2, 4, 6, 8, 10, or 12 following vaccination with H5N1 Viet Nam 1203 virus (1000×LD₅₀) (B) Percent weight loss over time in mice vaccinated with 50 ng rH5 alone or rH5 formulated with SE or GLA-SE at either 6 or 14 days prior to viral challenge. (C) Changes in general health in mice from (B) based on an observational scoring system; 0=normal; 1=questionable illness; 2=mild but definitive illness; 3=moderate illness; 4=severe, moribund.

FIGS. 4A-C. GLA-based rH5 vaccines provide durable protective immunity in mice following homologous and heterologous virus challenge. (A) Mice (5/group) were vaccinated with 50 ng of homologous (VN) rH5 alone or formulated as indicated and challenged 46 days later with H5N1 Viet Nam 1203 virus (1000×LD₅₀). Mice were monitored daily for weight change for two weeks following challenge. Each data point represents the mean weight loss per group +/− s.e.m. (B) Mice (5/group) were vaccinated with 50 ng of heterologous (Indo) rH5 alone or formulated as indicated and challenged 46 days later with H5N1 Viet Nam 1203 virus (1000×LD₅₀). Mice were monitored daily for weight change for two weeks following challenge. Each data point represents the mean weight loss per group +/− s.e.m. (C) Viral load was determined in animals vaccinated with homologous (VN) or heterologous (Indo) rH5 in the indicated formulations at day 3 or day 6 following challenge.

FIGS. 5A-B. A single injection with GLA-based rH5 vaccine protects ferrets from H5N1 infection. Animals (4/group) were injected once (IM) with 0.5 μg rH5 antigen alone or in the indicated formulations and then challenged on day 28 with an intranasal infusion of H5N1VN1203 (0.75×10⁶ pfu). (A) Percent change in body weight. Each data point represents the mean from 4 animals +/− s.e.m., with the exception of the GLA-SE and rH5 groups that present the results from, respectively, 1 and 2 survivors. (B) Changes in general health based on an observational scoring system; 0=normal; 1=questionable illness; 2=mild but definitive illness; 3=moderate illness; 4=severe, moribund.

### DETAILED DESCRIPTION

The present disclosure provides compositions for use as vaccines and methods of immunizing subjects with the vaccines, in which the vaccines comprise a recombinant hemagglutinin from a pre-pandemic or pandemic influenza virus and an adjuvant The vaccine provides protection against a pre-pandemic or pandemic influenza virus, and it generally elicits both humoral and cellular immunity and results in memory immune cells.

The vaccine and pharmaceutical compositions described herein comprise a hemagglutinin (HA) from a pre-pandemic or pandemic influenza virus and a DSLP adjuvant, e.g., an adjuvant according to formula (1), which may be GLA, and are directed to protect humans from this virus during pre-pandemic and pandemic stages. Moreover, the compositions provide benefits of augmenting immune responses against related viruses with fewer vaccinations (dose sparing) and/or lower dosages of HA than needed without adjuvant (dosage or antigen sparing), and eliciting cross-reactivity against related viruses.

### A. Hemagglutinin Preparation

1. Source of HA

At least four different influenza A viruses are currently of pre-pandemic or pandemic concern: H5N1, H1N1, H7N7, and H9N2.

Influenza A virus subtype H5N1 is a subtype of Influenza A virus, which can cause illness in humans and many other animal species. A highly pathogenic strain of H5N1; (HPAI H5N1), is the cause of “avian influenza” or “bird flu”. It is currently an avian disease, although it can infect humans, most or all of whom had extensive physical contact with infected birds. H5N1 is classified as a pre-pandemic virus because not all conditions of a pandemic have been met, most notably the virus does not spread easily and sustainably among humans.

Avian flu of the H5N1 type (herein called “H5N1”) first appeared in Asia and has been spreading globally. H5N1 viruses are evolving and can now be categorized based on the antigenic and sequence characteristics of their HA molecules into distinct clades and subclades. A “clade” refers to related organisms descended from a common ancestor. Since the re-emergence in 2003 of H5N1 infections in humans, several different clades have been isolated from the more than 300 cases of human disease. WHO (World Health Organization) has undertaken unifying a nomenclature system for the highly pathogenic H5N1 avian influenza viruses (Brown et al., Influenza Other Respi Viruses 3:59-62, 2009; Donis et al., Emerg Infect Dis. 14:e1, 2008; see also “Continuing progress towards a unified nomenclature system for the highly pathogenic H5N1 avian influenza viruses” at www.who.int). As of March 2009, 10 distinct clades of viruses (numbered 0-9) have been identified.

Clade 1 and Clade 2 viruses, which have been isolated mainly in SE Asia and Asia, are most often the subject of vaccine development. The first FDA-approved H5N1 vaccine was a conventional vaccine based on H5 clade 1 virus, but induced neutralizing titers at a protective level in only about half of the study participants, and, moreover, required two doses of a large amount of HA. A recombinant vaccine in testing gave similar results (Treanor et al., Vaccine 19:1732-1737). In addition to protecting more individuals, a vaccine capable of combating a pandemic needs both dose-sparing and dosage-sparing improvements.

There are approximately 1335 unique full-length H5 sequences in NCBI's “Influenza Virus Resource” (accessed 14 January 2010). Any of these H5 sequences can be used. The HA sequences may be from any clade or sub-clade. Most often, the HA will be from clades 1 or 2. WHO's reference H5 HA antigens are mainly in clade 2, but also include HAs in clades 1, 4, and 7 (“Antigenic and genetic characteristics of H5N1 viruses and candidate vaccine viruses developed for potential use in human vaccines” Feb 2009, accessed at www.who.int). The reference antigens include the antigens shown in the following Table(s).

| | | GenBank Acc No. |
|---|---|---|
| Virus name | Clade | or Reference* |
| A/VIETNAM/1203/2004 | 1 | ABW90135 |
| A/INDONESIA/5/2005 | 2.1 | ABP51969 |
| A/DUCK/HUNAN/795/2002 | 2.1 | ACA47835 |
| A/WHOOPER SWAN/MG/244/2005 | 2.2 | ACD68156 |
| A/TURKEY/65-596/2006 | 2.2 | ABQ58925 |
| A/BAR-HEADED | 2.2 | Chen et al. J. Virol. |
| GOOSE/QINGHAI/1A/2005XPR8 | | 80: 5976 (2006) |
| A/CHICKEN/INDIA/NIV-33487/2006 | 2.2 | ABQ45850 |
| A/EGYPT/321-NAMRU3/2007 | 2.2 | |
| A/CHICKEN/KOREA/GIMJE/2008 | 2.3.2 | |
| A/ANHUI/I/2005 | 2.3.4 | ABD28180 |
| A/ANHUI/I/2005XPR8 IBDC RG-6 | 2.3.4 | |
| A/CHICKEN/MALAYSIA/935/2006 | 2.3.4 | |
| A/JAPANESE WHITE EYE/HK/1038/2006 | 2.3.4 | ABJ96775 |
| A/GOOSE/GUIYANG/337/2006 | 4 | ABJ96698 |
| A/CHICKEN/VIETNAM/NCVD-016/2008 | 7 | ACO07033 |

*Sequences in all GenBank accessions and references are incorporated in their entirety.

Another subtype that is of pandemic concern is the H1N1 pandemic virus (“swine flu” virus), the type responsible for the 2009 flu pandemic and the Spanish flu of 1918. The strain causing the 2009 pandemic is called pandemic H1N1 2009 virus. As of 18 February 2010, over 900 non-redundant protein sequences of HA are available at NCBI and Influenza Research Database (fludb.org). For the United States, A/California/4/2009 (Accession No. ACP41105, incorporated in its entirety) and A/California/7/2009 (Accession No. ACQ55359, incorporated in its entirety) are strains used to make vaccines. HAs from other strains are also suitable.

Other potentially pandemic influenza viruses include H7N7 from the Netherlands and H9N2 from China. Since 2003, the Netherlands has been reporting outbreaks of another highly pathogenic avian influenza A virus (H7N7) in poultry. Confirmed infections of about 90 humans have occurred among poultry workers and families; antibodies to the virus were found widespread in people without contact with infected poultry but with close household contact to an infected individual, suggesting a high level of human-human transmission. H9N2 is another avian influenza virus found to have caused disease in humans in China. The National Institute of Allergy and Infectious Diseases has identified H9N2 as a potential pandemic virus. Some suitable strains include A/HK/1073/99 (H9N2) and A/NL/209/07 (H7N7).

Hemagglutinin proteins for pharmaceutical compositions and for vaccines can be full-length, but can also be a precursor protein, fragment, part of a fusion protein, or a peptide. A full-length protein refers to a mature protein; for example, in the case of a hemagglutinin protein, a mature protein is the form found in the virion (e.g., lacking a leader peptide, and may be cleaved from HA0 into HA1 and HA2). A precursor protein (pre-protein) is the nascent, translated protein before any processing occurs or a partially-processed protein. As part of a fusion protein, the HA protein may be present as a precursor or full-length protein or a protein fragment or a peptide. A fragment or peptide of a protein needs to be immunologic, containing one or more epitopes that elicit an immune response.

Peptides are chosen to complex with MHC molecules for binding to T cell receptors and are generally up to about 30 amino acids long, or up to about 25 amino acids long, or up to about 20 amino acids long, or up to about 15 amino acids long, up to about 12 amino acids long, up to about 9 amino acids long, up to about 8 amino acids long. In general, shorter peptides bind to or associate with MHC Class I molecules and longer peptides bind to or associate with MHC Class II molecules. Suitable peptides can be predicted using any of a number of bioinformatic programs and tested using well-known methods.

As disclosed herein, suitable proteins include precursor proteins, mature proteins, fragments, fusion proteins and peptides. More than one HA may be present in the composition. If multiple HA proteins are used, the HA proteins may be from the same virus or from different viruses. Furthermore, the multiple proteins may be present in the same form or as a mixture of these forms. For example, an HA protein may be present both as a mature protein and as a fragment.

Typically an HA in a pharmaceutical or vaccine composition will be other than a precursor protein because expression in a eukaryote of a glycoprotein that has a leader sequence will typically result in a mature protein, lacking the leader sequence (also known as a signal peptide). The length of the hydrophobic leader sequence of HA can vary somewhat among isolates, but is typically about 18 amino acids long. For recombinant expression however, a signal peptide may be part of the precursor protein. Signal peptides include the HA native sequence or others known in the art.

Protein fragments should be immunogenic. In some cases, the fragment(s) comprise immunodominant peptide sequences. Immunogenic peptide sequences are those recognized by B or T cells (e.g., CD4 or CD8 T cells). Peptide sequences can be identified by screening peptides derived from the complete sequence; generally using a series of overlapping peptides. A variety of assays can be used to determine if B or T cells recognize and respond to a peptide. For example, a chromium-release cytotoxicity assay (Kim et al., J Immunol 181:6604-6615, 2008, incorporated for assay protocol), ELISPOT assay, an intracellular cytokine staining assay and MHC multimer staining (Novak et al. J Clin Invest 104:R63-R67, 1999; Altman et al., Science 274:94-96, 1996), ELISA assays, other types of measurement of antibodies following immunization of mice with peptides coupled to a carrier are among suitable assays. Immunogenic peptides can also be predicted by bioinformatic software (“Immunoinformatics: Predicting immunogenicity in silico” Methods in Molecular Biology vol. 409, 2007). Some exemplary programs and databases include FRED (Feldhahn et al. Bioinformatics 15:2758-9, 2009), SVMHC (Dönnes and Kohlbacher, Nucleic Acids Res 34:W1940197, 2006), AntigenDB (Ansari et al., Nucleic Acids Res 38:D847-853, 2010), TEPITOPE (Bian and Hammer Methods 34:468-475, 2004).

HA can also be incorporated as part of a fusion protein. The other fusion partner or partners can be another HA protein or a non-HA protein, such as influenza neuraminidase. Some common reasons to use fusion proteins are to improve expression or aid in purification of the resulting protein. For example, a signal peptide sequence tailored for the host cell of an expression system can be linked to an HA protein or a tag sequence for use in protein purification can be linked, and subsequently cleaved if a cleavage sequence is also incorporated. Multiple peptide epitopes from one or more of the proteins can be fused or fragments from one or more of the proteins can be fused. Multiple peptide epitopes can be in any order.

Other suitable sources for HA in the compositions include virus-like particles (VLPs) containing HA (U.S. 2005009008, incorporated in its entirety), nucleic acids encoding HA (U.S. 2003045492; U.S. Pat. No. 7,537,768; WO 09092038; Smith et al. Vaccine Jan. 29, 2010, all of which are incorporated in their entirety), as well as attenuated and inactivated viruses (U.S. Pat. No. 6,022,726; U.S. Pat. No. 7,316,813; U.S. 2009010962; WO 99/57284, U.S. 2008254060; all of which are incorporated in their entirety).

2. Recombinant Synthesis—Vector Construction

HA proteins, including precursor proteins, fragments, fusion proteins and peptides may be produced in cultured cells or synthesized chemically. (“HA proteins” is hereby used herein to include all these forms.) Peptides, in particular, may be conveniently synthesized chemically, either using a machine (many are commercially available) or by hand. Alternatively, a variety of suitable expression systems, both prokaryotic and eukaryotic systems, are well known and may be used. Host cells often used and suitable for production of proteins include E. coli, yeast, insect, and mammalian. Expression vectors and host cells are commercially available (e.g., Invitrogen Corp., Carlsbad, Calif., USA) or may be constructed. An exemplary vector comprises a promoter and cloning site for the sequence encoding a protein of interest such that the promoter and sequence are operatively linked. Other elements may be present, such as a secretion signal sequence (sometimes called a leader sequence), a tag sequence (e.g., hexa-His), transcription termination signal, an origin of replication especially if the vector is replicated extra-chromosomally, and a sequence encoding a selectable product. Optional elements are arranged in the vector according to their purpose. Methods and procedures to transfect host cells are also well known.

Because HA is a glycosylated protein, most often the expression system of choice is a eukaryotic system that glycosylates protein, such as yeast (e.g., U.S. Pat. No. 5,856,123; U.S. RE35749; U.S. Pat. No. 4,925,791; also commercial systems such as PichiaPink™ Invitrogen, Calif. USA, K. lactis protein expression kit, Nebr., Mass., USA), mammalian cells and baculovirus (U.S. Pat. No. 4,745,051; U.S. Pat. No. 5,762,939; U.S. Pat. No. 5,858,368; U.S. Pat. No. 6,103,526; where all U.S. patent references identified herein are incorporated herein in their entirety).

Insect expression systems, in which a baculovirus comprising a coding sequence for HA infects insect cells that then express HA, are especially suitable expression systems. Expression in insect cells generally yields high concentration and amount of protein, produces proteins having eukaryotic post-translational modifications (e.g., glycosylation) and can be scaled up to production levels that generate protein sufficient for a pre-pandemic vaccine. Expression systems and methods are well known in the art; for example, there are many commercially available systems and service providers (e.g., Invitrogen, Carlsbad Calif.; Protein Sciences Meriden, Conn.; Clontech, Mountain View, Calif.; also see list of vendors and service providers at baculovirus.com).

In an exemplary insect expression system, the primary gene product is unprocessed, e.g., full length hemagglutinin, and is not secreted but remains associated with peripheral membranes of infected cells (U.S. Pat. No. 5,762,939). Several days post-infection, the recombinant HA (rHA) can be extracted from the peripheral membranes (U.S. Pat. No. 5,858,368, reference incorporated for extraction and purification of rHA). One suitable extraction method entails using a non-denaturing, non-ionic detergent or other well-known techniques. Expressed proteins may be used “as-is” or more typically, analyzed and further purified. Further purification can be done by, for example, affinity chromatography, gel chromatography, ion exchange chromatography or other equivalent methods know to those of ordinary skill in the art. rHA is purified to at least 80%, 85%, 90%, 95%, 98%, or 99% purity.

Typical procedures for determining purity or quantity include gel electrophoresis, Western blotting, mass spectrometry, and ELISA. Activity of proteins is generally assessed in a biological assay, such as those described in the Examples. If necessary or desired, proteins may be further purified. Many purification methods are well known and include size chromatography, anion or cation exchange chromatography, affinity chromatography, precipitation, immune precipitation, and the like. Intended use of the protein will typically determine the extent of purification, with use in humans requiring likely the highest level of purity.

### B. Adjuvant

The present invention provides compositions, kits, methods, etc. which include and/or utilize an adjuvant. The adjuvant is one or more compounds selected from the group denoted as DSLP. DSLP compounds share the features that they contain a disaccharide (DS) group formed by the joining together of two monosaccharide groups selected from glucose and amino substituted glucose, where the disaccharide is chemically bound to both a phosphate (P) group and to a plurality of lipid (L) groups. More specifically, the disaccharide may be visualized as being formed from two monosaccharide units, each having six carbons. In the disaccharide, one of the monosaccharides will form a reducing end, and the other monosaccharide will form a non-reducing end. For convenience, the carbons of the monosaccharide forming the reducing terminus will be denoted as located at positions 1, 2, 3, 4, 5 and 6, while the corresponding carbons of the monosaccharide forming the non-reducing terminus will be denoted as being located at positions 1′, 2′, 3′, 4′, 5′ and 6′, following conventional carbohydrate numbering nomenclature. In the DSLP, the carbon at the 1 position of the non-reducing terminus is linked, through either an ether ('O—) or amino (—NH—) group, to the carbon at the 6′ position of the reducing terminus. The phosphate group will be linked to the disaccharide, preferably through the 4′ carbon of the non-reducing terminus. Each of the lipid groups will be joined, through either amide (—NH—C(O—) or ester (—O—C(O)—) linkages to the disaccharide, where the carbonyl group joins to the lipid group. The disaccharide has 7 positions which may be linked to an amide or ester group, namely, positions 2′, 3′, and 6′ of the non-reducing terminus, and positions 1, 2, 3 and 4 of the reducing terminus.

A lipid group has at least six carbons, preferably at least 8 carbons, and more preferably at least 10 carbons, where in each case the lipid group preferably has no more than 24 carbons, preferably no more than 22 carbons, and more preferably no more than 20 carbons. In one aspect the lipid groups taken together provide 60-100 carbons, preferably 70 to 90 carbons. A lipid group may consist solely of carbon and hydrogen atoms, i.e., it may be a hydrocarbyl lipid group, or it may contain one hydroxyl group, i.e., it may be a hydroxyl-substituted lipid group, or it may contain an ester group which is, in turn, joined to a hydrocarbyl lipid or a hydroxyl-substituted lipid group through the carbonyl (—C(O)—)of the ester group, i.e., a ester substituted lipid. A hydrocarbyl lipid group may be saturated or unsaturated, where an unsaturated hydrocarbyl lipid group will have one double bond between adjacent carbon atoms.

The DSLP comprises 3, or 4, or 5, or 6 or 7 lipids. In one aspect, the DSLP comprises 3 to 7 lipids, while in another aspect the DSLP comprises 4-6 lipids. In one aspect, the lipid is independently selected from hydrocarbyl lipid, hydroxyl-substituted lipid, and ester substituted lipid. In one aspect, the 1, 4′ and 6′ positions are substituted with hydroxyl. In one aspect, the monosaccharide units are each glucosamine. The DSLP may be in the free acid form, or in the salt form, e.g., an ammonium salt.

In one aspect, the lipid on the DSLP is described by: the 3′ position is substituted with —O—(CO)—CH₂—CH(Rₐ)(—O—C(O)—R_{b}); the 2′ position is substituted with —NH—(CO)—CH₂—CH(Rₐ)(—O—C(O)—R_{b}); the 3 position is substituted with —O—(O)—CH₂—CH(OH)(Rₐ); the 2 position is substituted with —NH—(CO)—CH₂—CH(OH)(Rₐ); where each of Rₐ and R_{b} is selected from decyl, undecyl, dodecyl, tridecyl, tetradecyl, where each of these terms refer to saturated hydrocarbyl groups. In one embodiment, Rₐ is undecyl and R_{b} is tridecyl, where this adjuvant is described in, e.g., U.S. patent application publication 2008/0131466 as “GLA”. The compound wherein Rₐ is undecyl and R_{b} is tridecyl may be used in a stereochemically defined form, as available from, e.g., Avanti Polar Lipid as PHAD™ adjuvant.

In another aspect, the DSLP is a mixture of naturally-derived compounds known as 3D-MPL. 3D-MPL adjuvant is produced commercially in a pharmaceutical grade form by GlaxoSmithKline Company as their MPL™ adjuvant. 3D-MPL has been extensively described in the scientific and patent literature, see, e.g., Vaccine Design: the subunit and adjuvant approach, Powell M. F. and Newman, M. J. eds., Chapter 21 Monophosphoryl Lipid A as an adjuvant: past experiences and new directions by Ulrich, J. T. and Myers, K. R., Plenum Press, New York (1995) and U.S. Pat. No. 4,912,094.

In another aspect, the DSLP adjuvant may be described as comprising (i) a diglucosamine backbone having a reducing terminus glucosamine linked to a non-reducing terminus glucosamine through an ether linkage between hexosamine position 1 of the non-reducing terminus glucosamine and hexosamine position 6 of the reducing terminus glucosamine; (ii) an O-phosphoryl group attached to hexosamine position 4 of the non-reducing terminus glucosamine; and (iii) up to six fatty acyl chains; wherein one of the fatty acyl chains is attached to 3-hydroxy of the reducing terminus glucosamine through an ester linkage, wherein one of the fatty acyl chains is attached to a 2-amino of the non-reducing terminus glucosamine through an amide linkage and comprises a tetradecanoyl chain linked to an alkanoyl chain of greater than 12 carbon atoms through an ester linkage, and wherein one of the fatty acyl chains is attached to 3-hydroxy of the non-reducing terminus glucosamine through an ester linkage and comprises a tetradecanoyl chain linked to an alkanoyl chain of greater than 12 carbon atoms through an ester linkage. See, e.g., U.S. patent application publication 2008/0131466.

In another aspect, the adjuvant may be a synthetic disaccharide having six lipid groups as described in U.S. patent application publication 2010/0310602.

In another aspect, the adjuvant used in the present invention may be identified by chemical formula (1):

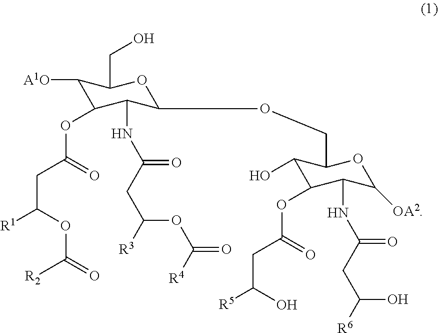

In chemical formula (1), the moieties A¹ and A² are independently selected from the group of hydrogen, phosphate, and phosphate salts. Sodium and potassium are exemplary counterions for the phosphate salts. The A¹O-group, which is preferably a phosphate group, is bonded to the disaccharide at the 4′ position of the non-reducing terminus. The non-reducing terminus is bonded through its 1 position to an ether group, which in turn is bonded to the 6′ position of the reducing terminus. The compounds of chemical formula (1) have six lipid groups that each incorporate one of the moieties R¹, R², R³, R⁴, R⁵, and R⁶, where these R groups are independently selected from the group of hydrocarbyl having 3 to 23 carbons, represented by C₃-C₂₃. For added clarity it will be explained that when a moiety is “independently selected from” a specified group having multiple members, it should be understood that the member chosen for the first moiety does not in any way impact or limit the choice of the member selected for the second moiety. The carbon atoms to which R¹, R³, R⁵ and R⁶ are joined are asymmetric, and thus may exist in either the R or S stereochemistry. In one embodiment all of those carbon atoms are in the R stereochemistry, while in another embodiment all of those carbon atoms are in the S stereochemistry.

“Hydrocarbyl” refers to a chemical moiety formed entirely from hydrogen and carbon, where the arrangement of the carbon atoms may be straight chain or branched, noncyclic or cyclic, and the bonding between adjacent carbon atoms maybe entirely single bonds, i.e., to provide a saturated hydrocarbyl, or there may be double or triple bonds present between any two adjacent carbon atoms, i.e., to provide an unsaturated hydrocarbyl, and the number of carbon atoms in the hydrocarbyl group is between 3 and 24 carbon atoms. The hydrocarbyl may be an alkyl, where representative straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while branched alkyls include isopropyl, sec-butyl, isobutyl, tert-butyl, isopentyl, and the like. Representative saturated cyclic hydrocarbyls include cyclopropyl, cyclobutyl, cyclopentyl, cyclohexyl, and the like; while unsaturated cyclic hydrocarbyls include cyclopentenyl and cyclohexenyl, and the like. Unsaturated hydrocarbyls contain at least one double or triple bond between adjacent carbon atoms (referred to as an “alkenyl” or “alkynyl”, respectively, if the hydrocarbyl is non-cyclic, and cycloalkeny and cycloalkynyl, respectively, if the hydrocarbyl is at least partially cyclic). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

The DSLP adjuvant may be obtained by synthetic methods known in the art, for example, the synthetic methodology disclosed in PCT International Publication No. WO 2009/035528, which is incorporated herein by reference, as well as the publications identified in WO 2009/035528, where each of those publications is also incorporated herein by reference. A chemically synthesized DSLP adjuvant, e.g., the adjuvant of formula (1), can be prepared in substantially homogeneous form, which refers to a preparation that is at least 80%, preferably at least 85%, more preferably at least 90%, more preferably at least 95% and still more preferably at least 96%, 97%, 98% or 99% pure with respect to the DSLP molecules present, e.g., the compounds of formula (1). Determination of the degree of purity of a given adjuvant preparation can be readily made by those familiar with the appropriate analytical chemistry methodologies, such as by gas chromatography, liquid chromatography, mass spectroscopy and/or nuclear magnetic resonance analysis. DSLP adjuvants obtained from natural sources are typically not easily made in a chemically pure form, and thus synthetically prepared adjuvants are preferred adjuvants of the present invention. As mentioned previously, certain of the adjuvants may be obtained commercially. A preferred adjuvant is Product No. 699800 as identified in the catalog of Avanti Polar Lipids, Alabaster Ala., see E1 in combination with E10, below.

In various embodiments of the invention, the adjuvant has the chemical structure of formula (1) but the moieties A¹, A², R¹, R², R³, R⁴, R⁵, and R⁶ are selected from subsets of the options previously provided for these moieties, where these subsets are identified below by E1, E2, etc.
E1: A₁ is phosphate or phosphate salt and A₂ is hydrogen.
E2: R¹, R³, R⁵ and R⁶ are C₃-C₂₁ alkyl; and R² and R⁴ are C₅-C₂₃ hydrocarbyl.
E3: R¹, R³, R⁵ and R⁶ are C₅-C₁₇ alkyl; and R² and R⁴ are C₇-C₁₉ hydrocarbyl.
E4: R¹, R³, R⁵ and R⁶ are C₇-C₁₅ alkyl; and R² and R⁴ are C₉-C₁₇ hydrocarbyl.
E5: R¹, R³, R⁵ and R⁶ are C₉-C₁₃ alkyl; and R² and R⁴ are C₁₁-C₁₅ hydrocarbyl.
E6: R¹, R³, R⁵ and R⁶ are C₉-C₁₅ alkyl; and R² and R⁴ are C₁₁-C₁₇hydrocarbyl.
E7: R¹, R³, R⁵ and R⁶ are C₇-C₁₃ alkyl; and R² and R⁴ are C₉-C₁₅ hydrocarbyl.
E8: R¹, R³, R⁵ and R⁶ are C₁₁-C₂₀ alkyl; and R² and R⁴ are C₁₂-C₂₀ hydrocarbyl.
E9: R¹, R³, R⁵ and R⁶ are C₁₁ alkyl; and R² and R⁴ are C₁₃ hydrocarbyl.
E10: R¹, R³, R⁵ and R⁶ are undecyl and R² and R⁴ are tridecyl.

In certain options, each of E2 through E10 is combined with embodiment E1, and/or the hydrocarbyl groups of E2 through E9 are alkyl groups, preferably straight chain alkyl groups.

The DSLP adjuvant, e.g., the adjuvant of formula (1), may be formulated into a pharmaceutical composition, optionally with a co-adjuvant, each as discussed below. In this regard reference is made to U.S. Patent Publication No. 2008/0131466 which provides formulations, e.g., aqueous formulation (AF) and stable emulsion formulations (SE) for GLA adjuvant, where these formulations may be utilized for any of the DSLP adjuvants, including the adjuvants of formula (1).

The present invention provides that the DSLP adjuvant, e.g., the adjuvant of formula (1), may be utilized in combination with a second adjuvant, referred to herein as a co-adjuvant. In three embodiments of the invention, the co-adjuvant may be a delivery system, or it may be an immunopotentiator, or it may be a composition that functions as both a delivery system and an immunopotentiator, see, e.g., O'Hagan D T and Rappuoli R., Novel approaches to vaccine delivery, Pharm. Res. 21(9):1519-30 (2004). The co-adjuvant may be an immunopotentiator that operates via a member of the Toll-like receptor family biomolecules. For example, the co-adjuvant may be selected for its primary mode of action, as either a TLR4 agonist, or a TLR8 agonist or a TLR9 agonist. Alternatively, or in supplement, the co-adjuvant may be selected for its carrier properties, e.g., it may be an emulsion, a liposome, a microparticle, or alum.

In one aspect, the co-adjuvant is alum, where this term refers to aluminum salts, such as aluminum phosphate (AIPO₄) and aluminum hydroxide (Al(OH)₃). When alum is used as the co-adjuvant, the alum may be present, in a dose of vaccine, in an amount of about 100 to 1,000 μg, or 200 to 800 μg, or 300 to 700 μg or 400 to 600 μg. The DSLP adjuvant, e.g., the adjuvant of formula (1), is typically present in an amount less than the amount of alum, in various aspects the DSLP adjuvant, e.g., the adjuvant of formula (1), on a weight basis, is present at 0.1-1%, or 1-5%, or 1-10%, or 1-100% relative to the weight of alum.

In one aspect, the co-adjuvant is an emulsion having vaccine adjuvanting properties. Such emulsions include oil-in-water emulsions. Freund's incomplete adjuvant (IFA) is one such adjuvant. Another suitable oil-in-water emulsion is MF-59™ adjuvant which contains squalene, polyoxyethylene sorbitan monooleate (also known as Tween™ 80 surfactant) and sorbitan trioleate. Squalene is a natural organic compound originally obtained from shark liver oil, although it is also available from plant sources (primarily vegetable oils), including amaranth seed, rice bran, wheat germ, and olives. Other suitable adjuvants are Montanide™ adjuvants (Seppic Inc., Fairfield N.J.) including Montanide™ ISA 50V which is a mineral oil-based adjuvant, Montanide™ ISA 206, and Montanide™ IMS 1312. While mineral oil may be present in the co-adjuvant, in one embodiment the oil component(s) of the vaccine compositions of the present invention are all metabolizable oils.

Examples of immunopotentiators which may be utilized in the practice of the present invention as co-adjuvants include: 3D-MPL or MPL™ adjuvant, MDP and derivatives, oligonucleotides, double-stranded RNA, alternative pathogen-associated molecular patterns (PAMPS); saponins, small-molecule immune potentiators (SMIPs), cytokines, and chemokines.

In one embodiment the co-adjuvant is 3D-MPL or MPL™ adjuvant, where the latter is commercially available from GlaxoSmithKline, although it was originally developed by Ribi ImmunoChem Research, Inc. Hamilton, Mont. See, e.g., Ulrich and Myers, Chapter 21 from Vaccine Design: The Subunit and Adjuvant Approach, Powell and Newman, eds. Plenum Press, New York (1995). Related to MPL™ adjuvant, and also suitable as co-adjuvants in the present invention, are AS02™ adjuvant and ASO4™ adjuvant. AS02™ adjuvant is an oil-in-water emulsion that contains both MPL™ adjuvant and QS-21™ adjuvant (a saponin adjuvant discussed elsewhere herein). ASO4™ adjuvant contains MPL™ adjuvant and alum. MPL™ adjuvant is prepared from lipopolysaccharide (LPS) of Salmonella Minn. R595 by treating LPS with mild acid and base hydrolysis followed by purification of the modified LPS, as described more completely in the article by Ulrich and Myers.

In one embodiment, the co-adjuvant is a saponin such as those derived from the bark of the Quillaja saponaria tree species, or a modified saponin, see, e.g., U.S. Pat. Nos. 5,057,540; 5,273,965; 5,352,449; 5,443,829; and 5,560,398. The product QS-21 ™ adjuvant sold by Antigenics, Inc. Lexington, Mass. is an exemplary saponin-containing co-adjuvant that may be used with the DSLP adjuvant, e.g., the adjuvant of formula (1). Related to the saponins is the ISCOM™ family of adjuvants, originally developed by Iscotec (Sweden) and typically formed from saponins derived from Quillaja saponaria or synthetic analogs, cholesterol, and phospholipid, all formed into a honeycomb-like structure.

In one embodiment, the co-adjuvant is a cytokine which functions as a co-adjuvant, see, e.g., Lin R. et al. Clin. Infec. Dis. 21(6):1439-1449 (1995); Taylor, C. E., Infect. Immun. 63(9):3241-3244 (1995); and Egilmez, N. K., Chap. 14 in Vaccine Adjuvants and Delivery Systems, John Wiley & Sons, Inc. (2007). In various embodiments, the cytokine may be, e.g., granulocyte-macrophage colony-stimulating factor (GM-CSF); see, e.g., Change D. Z. et al. Hematology 9(3):207-215 (2004), Dranoff, G. Immunol. Rev. 188:147-154 (2002), and U.S. Pat. No. 5,679,356; or an interferon, such as a type I interferon, e.g., interferon-α (IFN-α) or interferon-β (IFN-β), or a type II interferon, e.g., interferon-γ (IFN-γ), see, e.g., Boehm, U. et al. Ann. Rev. Immunol. 15:749-795 (1997); and Theofilopoulos, A. N. et al. Ann. Rev. Immunol. 23:307-336 (2005); an interleukin, specifically including interleukin-1α (IL-1α), interleukin-1β (IL-1β), interleukin-2 (IL-2); see, e.g., Nelson, B. H., J. Immunol. 172(7):3983-3988 (2004); interleukin-4 (IL-4), interleukin-7 (IL-7), interleukin-12 (IL-12); see, e.g., Portielje, J. E., et al., Cancer Immunol. Immunother. 52(3): 133-144 (2003) and Trinchieri. G. Nat. Rev. Immunol. 3(2):133-146 (2003); interleukin-15 (II-15), interleukin-18 (IL-18); fetal liver tyrosine kinase 3 ligand (FIt3L), or tumor necrosis factor a (TNFα). The DSLP adjuvant, e.g., the adjuvant of formula (1), may be co-formulated with the cytokine prior to combination with the vaccine antigen, or the antigen, DSLP adjuvant, e.g., the adjuvant of formula (1) and cytokine co-adjuvant may be formulated separately and then combined.

In one embodiment, the co-adjuvant is unmethylated CpG dinucleotides, optionally conjugated to the flu antigen described herein.

When a co-adjuvant is utilized in combination with the DSLP adjuvant, e.g., the adjuvant of formula (1), the relative amounts of the two adjuvants may be selected to achieve the desired performance properties for the vaccine composition which contains the adjuvants, relative to the antigen alone. For example, the adjuvant combination may be selected to enhance the antibody response of the antigen, and/or to enhance the subject's innate immune system response. Activating the innate immune system results in the production of chemokines and cytokines, which in turn activate an adaptive (acquired) immune response. An important consequence of activating the adaptive immune response is the formation of memory immune cells so that when the host re-encounters the antigen, the immune response occurs quicker and generally with better quality.

The combination of vaccine adjuvants can be used strategically to regulate both the quantity and quality of immune responses needed for controlling pre-pandemic and pandemic flu. Water and oil emulsion-based adjuvants induce Th2 T cell immunity. Their use is important for driving production of neutralizing antibodies that protect against viral infection, however, these emulsions are not effective in stimulating cell mediated immunity. Given a pandemic outbreak, inducement of Th1 T cells is critical to limit disease progression within the host and to reduce viral transmission within the population. Th1 T cells play a direct antiviral role against human influenza viruses via production of IFN-γ and TNFα (Kannaganat et al., 181:8468-8476, 2007). They also regulate the expansion, maintenance and recall of anti-viral CD8 cytotoxic T-lymphocytes that are critical for viral clearance, as well as stimulate production of a subclass of antibodies (IgG2a in mice) that are protective against influenza, even in the absence of high virus-neutralizing activity (Huber et al. Clin Vaccine Immunol 13:981-990, 2006). The most direct method for inducing Th1 responses involves activation of Toll-like receptors (TLR) that recognize and bind pathogen derived sugars, proteins, lipids, and nucleic acids. Toll-like receptors stimulate dendritic cell maturation and are required for normal innate and adaptive immunity. While the DSLP adjuvant, e.g., the adjuvant of formula (1) alone may achieve each of these various goals, the present invention provides, in one embodiment, that adjuvant combinations may be utilized in combination with a pandemic flu antigen to achieve these goals. However, in a separate embodiment, the only adjuvant present in the vaccine is DSLP adjuvant, e.g., the adjuvant of formula (1) including the various embodiments thereof, where GLA is a preferred DSLP adjuvant of formula (1).

Moreover, GLA in an oil-in-water emulsion significantly enhanced the immunogenicity of Fluzone vaccine in mice, as measured by increases in antigen-specific antibodies and HAI titers, dose-sparing, and broadened cross-reactivity to antigen-drifted strains of influenza. In these same experiments, GLA induced Th1 T cell responses as indicated by a dramatic increase in antigen-specific IgG2a titers and IFNγ production.

### C. Pharmaceutical Compositions, Vaccines, and Their Uses 1. Formulation

Pharmaceutical compositions comprise a recombinant hemagglutinin (HA) from a pre-pandemic or pandemic influenza and a DSLP adjuvant, e.g., an adjuvant of formula (1), such as GLA. The DSLP adjuvant, e.g., the adjuvant of formula (1), may be formulated in an oil-in-water emulsion, as an aqueous solution, or in a liposome, as three examples. The compositions may additionally comprise other proteins, such as neuraminidase of the pre-pandemic or pandemic influenza, other adjuvants such as aluminum salts (e.g., alum) or saponin and saponin derivatives, excipients such as alpha-tocopherol or derivative, carriers, buffers, stabilizers, binders, preservatives such as thimerosal, surfactants, etc.

The DSLP adjuvant, e.g., the adjuvant of formula (1), can be used alone or formulated in an oil-in-water emulsion in which the adjuvant is incorporated in the oil phase, as two options. When used alone, i.e., without benefit of being combined with an emulsion, the adjuvant is typically completely oil-free or is present in a composition that contains less than about 1% v/v oil. In order to prepare an oil-free composition, water, adjuvant (e.g., GLA is a preferred adjuvant) and a surfactant, e.g., a phospholipid, e.g., 1-palmitoyl-2-oleoyl-sn-glycero-3-phosphocholine (POPC) may be combined. The composition may be prepared by adding a solution of ethanol and POPC to a pre-weighed amount of GLA. This wetted GLA is sonicated for 10 minutes to disperse the GLA as much as possible. The GLA is then dried under nitrogen gas. The dried GLA and POPC are reconstituted with WFI (water-for-injection) to the correct volume. This solution is sonicated at 60° C. for 15-30 minutes until all the GLA and POPC are in solution. For long term storage, GLA-AF formulations must be lyophilized. The lyophilization process consists of adding glycerol to the solution until it is 2% of the total volume. Then the solution is placed in vials in 1-10 mL amounts. The vials are run through a lyophilization process which consists of freezing the solution and then putting it under vacuum to draw off the frozen water by sublimation.

When the adjuvant will be combined with an oil, then for use in humans, the oil is preferably metabolizable. The oil may be any vegetable oil, fish oil, animal oil or synthetic oil; the oil should not be toxic to the recipient and is capable of being transformed by metabolism. Nuts (such as peanut oil), seeds, and grains are common sources of vegetable oils. Particularly suitable metabolizable oils include squalene (2,6,10,15,19,23-hexamethyl-2,6,10,14,18,22-tetracosahexane), an unsaturated oil found in many different oils, and in high quantities in shark-liver oil. Squalene is an intermediate in the biosynthesis of cholesterol. In addition, the oil-in-water emulsions typically comprise an antioxidant, such as alpha-tocopherol (vitamin E, U.S. Pat. No. 5,650,155, U.S. Pat. No. 6,623,739). Stabilizers, such as a triglyceride, ingredients that confer isotonicity, and other ingredients may be added.

The average size of the oil droplets is typically less than 1 micron, may be in the range of 30-600 nm, and usually about 80 to about 120 nm or less than about 150 nm. Oil droplet size may be measured by photon correlation spectroscopy. Typically, at least about 80% of the oil droplets should be within the desired ranges, or at least about 90% or at least about 95%. The fraction of oil in the emulsions is generally in the range of 2 to 10% (e.g., about 2%, about 3%, about 4%, about 5%, about 6%, about 7%, about 8%, about 9% and about 10%); the fraction of an anti-oxidant, such as alpha-tocopherol from about 2 to about 10%, and of a surfactant from about 0.3 to 3%. Preferably the ratio of oil:alpha tocopherol is equal or less than 1 as this provides a more stable emulsion. Sorbitan trioleate (e.g., Span® 85) may also be present at a level of about 1%. In some cases it may be advantageous that the vaccines of the present invention will further contain a stabilizer.

The method of producing oil in water emulsions is well known to the person skilled in the art. Commonly, the method comprises mixing the oil phase with a surfactant, such as phosphatidylcholine, block co-polymer, or a TWEEN80® solution, followed by homogenization using a homogenizer. For instance, a method that comprises passing the mixture once, twice or more times through a syringe needle would be suitable for homogenizing small volumes of liquid. Equally, the emulsification process in a microfluidiser (M110S microfluidics machine, maximum of 50 passes, for a period of 2 min at maximum pressure input of 6 bar (output pressure of about 850 bar)) could be adapted to produce smaller or larger volumes of emulsion. This adaptation could be achieved by routine experimentation comprising the measurement of the resultant emulsion until a preparation was achieved with oil droplets of the required diameter. Other equipment or parameters to generate an emulsion may also be used.

An exemplary oil-in-water emulsion using squalene is known as “SE” and comprises squalene, glycerol, phosphatidylcholine or lecithin or other block co-polymer as a surfactant in an ammonium phosphate buffer pH 5.1 with alpha-toceraphol. When GLA is used as the DSLP, the resulting composition is referred to herein as GLA-SE. To make such a composition, GLA (100 micrograms; Avanti Polar Lipids, Inc., Alabaster, Ala.; product number 699800) is emulsified in squalene (34.3 mg) with glycerol (22.7 mg), phosphotidylcholine or lecithin (7.64 mg), Pluronic® F-68 (BASF Corp., Mount Olive, N.J.) or similar block co-polymer (0.364 mg) in 25 millimolar ammonium phosphate buffer (pH=5.1) optionally using 0.5 mg D,L-alpha-tocopherol as an antioxidant. The mixture is processed under high pressure until an emulsion forms that does not separate and that has an average particle size of less than 180 nm. The emulsion is then sterile-filtered into glass unidose vials and capped for longer term storage. This preparation may be used for at least three years when stored at 2-8° C. Other oil-containing compositions that include a DSLP and a protein as described herein may be prepared in analogy to those compositions prepared as described in U.S. Pat. Nos. 5,650,155; 5,667,784; 5,718,904; 5,961,970; 5,976,538; 6,630,161; and 6,572,861.

Some particular compositions and vaccines comprise rH5 from the pre-pandemic H5N1 virus. Various forms of rHA suitable for a vaccine are discussed above; briefly, these include a full-length rHA, a fragment of rHA, a fusion protein comprising rHA, or a peptide. Furthermore, more than one rHA can be used together or with other proteins, such as neuraminidase, may be in the compositions and vaccines. More than one rHA form, or rHA sequence, or both, may be combined in a composition of the present invention.

The amount of rHA protein in a vaccine is typically a low dose e.g. from about 0.1 μg to about 15 μg. The low amount may be any of 0.1, 0.5, 1, 2, 3, 4, 5, 6, 7, 8, 9, 10, 11, 12, 13, 14, or 15 μg of rHA protein. The low amount of rHA protein may be as low as practically feasible provided that it allows formulation of vaccine that meets an international (e.g. EU or FDA) criterion for efficacy, as detailed below. The dose will typically be determined by activity, number of intended administrations, and the size and condition of the subject.

The proteins may be provided as a solution, but can also be provided in dry form (e.g., desiccated), in which case, a user adds any necessary liquid. Typically, additives such as buffers, stabilizers, preservatives, excipients, carriers, and other non-active ingredients will also be present. Additives are typically pharmaceutically acceptable and bio-compatible.

The DSLP adjuvant, e.g., the adjuvant of formula (1), may be provided as a solution, desiccated, or emulsified, and conveniently is provided as a stable oil-in-water emulsion. In addition to the DSLP adjuvant, e.g., the adjuvant of formula (1), the adjuvant-containing compositions may also comprise buffers, stabilizers, excipients, preservatives, carriers, or other non-active ingredients. Additives are typically pharmaceutically acceptable and bio-compatible. Additional adjuvants may be present, as described in more detail elsewhere herein. These co-adjuvants include, 2′-5′ oligo A, bacterial endotoxins, RNA duplexes, single stranded RNA, lipoprotein, peptidoglycan, flagellin, CpG DNA, lipopolysaccharide, MPA (monophosphoryl lipid A), 3-O-deacylated MPL, lipopolysaccharide, QS21 (a saponin), aluminium hydroxide (“alum”) and other mineral salts, oil emulsions (e.g., MF59™, R848 and other imidazoquinolines, virosomes and other particulate adjuvants (see, Vogel and Powell, “A compendium of vaccine adjuvants and excipients” Pharm Biotechnol 6:141-228, 1995; incorporated in its entirety).

The amount DSLP adjuvant, e.g. the adjuvant of formula (1), e.g., GLA, that is used in a dose of composition of the present invention (where a dose is an amount of composition administered to the subject in need thereof) that also contains antigen, useful as a vaccine, is in one embodiment about 0.5 μg to about 50 μg, in another embodiment is about 1.0 μg to 25 μg, and in various other embodiments of the present invention may be about 1 μg, about 2 μg, about 2.5 μg, about 5 μg, about 7.5 μg, about 10 μg, about 15 μg, about 20 μg or about 25 μg. The total volume of composition in a dose will typically range from 0.5 mL to 1.0 mL. An emulsion, such as SE, may be present in the composition, where the oil component(s) of the emulsion constitutes, in various embodiments, at about 0.1%, about 0.5%, about 1.0%, about 1.5%, about 2%, about 2.5%, about 3%, about 4%, about 5%, about 7.5% or about 10% of the total volume of the composition.

DSLP adjuvant, e.g., the adjuvant of formula (1), and protein may be provided in separate containers and mixed on-site or pre-mixed. In addition, the protein may be presented in separate containers or combined in a single container. A container can be a vial, ampoule, tube, or well of a multi-well device, reservoir, syringe or any other kind of container. The container or containers may be provided as a kit. If one or more of the containers comprises desiccated ingredients the liquids for reconstitution may be provided in the kit as well or provided by the user. The amount of solution in each container or that is added to each container is commensurate with the route of administration and how many doses are in each container. A vaccine given by injection is typically from about 0.1 ml to about 1.0 ml, while a vaccine that is given orally may be a larger volume, from about 1 ml to about 10 ml for example. Suitable volumes may also vary according to the size and age of the subject.

The compositions are generally provided sterile. Typical sterilization methods include filtration, irradiation and gas treatment.

2. Administration

The vaccine can be administered by any suitable delivery route, such as intradermal, mucosal (e.g., intranasal, oral), intramuscular, subcutaneous, sublingual, rectal, vaginal. Other delivery routes are well known in the art.

The intramuscular route is one suitable route for the vaccine composition. Suitable i.m. delivery devices include a needle and syringe, a needle-free injection device (for example Biojector, Bioject, Oreg. USA), or a pen-injector device, such as those used in self-injections at home to deliver insulin or epinephrine. Intradermal and subcutaneous delivery are other suitable routes. Suitable devices include a syringe and needle, syringe with a short needle, and jet injection devices.

The vaccine may be administered by a mucosal route, e.g., intranasally. Many intranasal delivery devices are available and well known in the art. Spray devices are one such device. Oral administration is as simple as providing a solution for the subject to swallow.

Vaccine may be administered at a single site or at multiple sites. If at multiple sites, the route of administration may be the same at each site, e.g., injection in different muscles, or may be different, e.g., injection in a muscle and intranasal spray. Furthermore, the vaccine may be administered at a single time point or multiple time points. Generally if administered at multiple time points, the time between doses has been determined to improve the immune response.

Vaccine is administered at a dose sufficient to effect a beneficial immune response to prevent or lessen symptoms of disease or infection. One indicator of a beneficial response is development of antibodies to the pre-pandemic or pandemic influenza virus, and also particularly, development of neutralizing antibodies. Other indicators include an increased amount or function or frequency of CD8 or CD4 T cells responsive to virus, and a reduction in virus transmission.

Many well known procedures are available to detect and quantify antibodies, including ELISA and inhibition of virus infection (neutralization) assays. In one implementation, the ELISA assay is performed by coating wells of a multi-well plate with HA protein, capturing HA-specific antibody from serum onto the plates, detecting the HA-specific antibody with labeled anti-human antibody, followed by a readout of the label. Label can be radioactive, but is more usually an enzyme, such as horse radish peroxidase, that converts a substrate to one that can be detected colorimetrically.

An exemplary influenza neutralization assay is based on a plaque assay in which neutralizing antibody is detected by inhibition of infectivity. The virus neutralization test is a highly sensitive and specific assay for identifying influenza virus-specific antibodies in animals and humans. The neutralization test is performed in two stages: (1) a virus-antibody reaction step, in which the virus is mixed with dilutions of serum and incubated to allow antibodies to bind to the virus, and (2) an inoculation step, in which the mixture is inoculated into the appropriate host system (e.g. cell cultures such as MDCK cells, embryonated eggs, or animals). When cells are used, virally infected cells are detected the next day in a microneutralization assay. The cells are fixed and the presence of influenza A virus nucleoprotein (NP) in infected cells is detected by ELISA. The detection of NP indicates the absence of neutralizing antibodies at that serum dilution. The absence of infectivity constitutes a positive neutralization reaction and indicates the presence of virus-specific antibodies in the serum sample. (“Influenza Virus Microneutralization Assay”, CDC publication, LP-004, R-2 (K Hancock) Effective October 19, 2009.) Other neutralization tests for influenza viruses are based on the inhibition of cytopathic effect (CPE) formation in MDCK cell cultures (see, Sidwell and Smee, Antiviral Res. 48:1-16, 2000).

Another well-known assay is a hemagglutination-inhibition assay, which assesses immune responses to influenza virus HA. The HA protein on the virus surface agglutinates erythrocytes (red blood cells, RBCs). When antibodies to HA bind to HA, agglutination is inhibited. In general, a standardized amount of HA antigen is mixed with serum samples that have been serially diluted, erythrocytes are added, and the extent of agglutination is assessed. Non-specific inhibitors of agglutination in serum are first removed by adsorption of serum on RBCs (“Serologic Detection of Human Influenza Virus Infections by Hemagglutination-Inhibition Assay Using Turkey RBCs”, CDC publication, LP-003, R-1 (K Hancock) Effective October 2, 2009).

The type and sub-type of antibodies produced may also be determined. Assays for determining IgM, IgG and IgG sub-types, and IgA are well-known in the art. One commonly used assay is ELISA. Briefly, microtiter plates are coated with antigen, e.g., rHA or inactivated whole virus. After blocking in a solution containing protein (e.g., 1% bovine serum albumin), serial dilutions of serum samples are added to the wells, followed by treatment with immunoglobulin isotype specific secondary antibody. Either the anti-isotype antibody is labeled or a labeled antibody-binding molecule is added. The amount of label is determined.

A criterion to the FDA for a pre-pandemic vaccine is the ability to induce immunity that is cross-reactive, meaning that the vaccine induces immunity to genetically distinct viruses from different clades and sub-clades. Cross-reactivity can be tested by any of the antibody assays described herein or by others known in the art. In an exemplary assay, sera are tested for antibodies to HA from a variety of viruses, including the virus containing the immunizing HA. For these assays, HA protein or whole virus (preferably inactivated) can be used.

Assays for T cell function include IFN-γ ELISPOT and ICS (intracellular cytokine staining). By measuring cytokine production for several cytokines Th1/Th2 profiles can be established. In particular, a desirable pattern is increases in IFN-γ and IL-2 production and reduced IL-5 and IL-4 production. ELISPOT assay detecting interferon-gamma is widely used to quantize CD4 and CD8 T cell responses to candidate vaccines. The ELISPOT assay is based on the principle of the ELISA detecting antigen-induced secretion of cytokines trapped by an immobilized antibody and visualized by an enzyme-coupled second antibody. ICS is a routinely used method to quantify cytotoxic T cells by virtue of cytokine expression following stimulation with agonists, such as antibodies to T cell surface molecules or peptides that bind MHC Class molecules. Exemplary procedures of ICS and ELISPOT are described in the examples below.

Antigen-specific T cell function can also be measured. Influenza-specificspecific-CD4+T cells that co-express IFNγ, IL-2 and TNF have better functional activity and costimulatory potential relative to cells that produce a single cytokine. Thus, the induction of multiple cytokine-producing CD4+ T cells is desirable. Antigen-specific T-cell stimulation assays may be used to estimate the frequency of CD4 T cells that produce IFNγ, IL-2, TNFa, and combinations thereof by flow cytometry. The addition of IL-5 to this assay can be used to distinguish Th1 vs Th2 CD4+ cells. A time course experiment at 3, 6, 12, and 24 weeks post-immunization is performed to determine long-lasting T cell responses. Flow cytometry can also be used to measure and distinguish the generation of effector memory CD4+ T cells (TEM: CD4+CD62L-CCR7-IFNγ+) and central memory CD4 T (TCM:CD4+CD62L+CCR7+IL2+IFNγ+/−) cells. Vaccine formulations that induce production of IFNγ, TNF and IL-2 and increase CD4CM are desirable. Cytotoxic CD8+ T cells also play a role in clearing virus load and limiting disease progression. Vaccines that elicit antigen-specific CD8+ T cells are desirable.

In one aspect the present invention provides a method of immunizing a population of people against a pre-pandemic or pandemic influenza virus, where those people are potentially going to be exposed to the virus. The method comprises administering a single injection of a pharmaceutical composition, where this single injection achieves seroconversion in at least 50% of the population that receives the single injection. The pharmaceutical compositions comprises (a) a recombinant hemagglutinin (rHA) from a pre-pandemic or pandemic influenza virus and (b) a DSLP adjuvant, e.g., an wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons. In various embodiments, the composition does not include an emulsion, or does not include any oil, e.g., squalene. Oil-free compositions are viewed by some health professional as being prone to less side-effects. For instance, there is mounting concern that emulsions tend to make a vaccine composition reactogenic in that, when the oil is present, the vaccine can cause stinging and pain to the subject receiving the injection. Another advantage of an emulsion-free composition is that in typical use, vaccine compositions are made from two components: an antigen-containing composition and an adjuvant-containing composition, where these two compositions are mixed in order to prepare the final vaccine. The stability of each of the compositions is preferably high over a long term. One problem with emulsions as a carrier for an adjuvant or antigen is that emulsions tend to be unstable over the long term, or require special conditions or chemicals to maintain their stability. The present invention, in one embodiment, provides oil-free, e.g., emulsion-free compositions that exhibit good stability and good efficacy. In a preferred embodiment, the DSLP adjuvant is GLA, and more preferably is GLA (or other DSLP adjuvant) in an oil-free carrier, which can be stored for a long time, e.g., in a lyophilized form which consists only of adjuvant and surfactant, e.g., phospholipid, and then mixed with a carrier and an antigen to provide an effective vaccine.

Other desirable aspects of a vaccine include dose- and dosage-sparing properties. Dosage-sparing means that fewer doses than usual may be administered to a person to still raise a desired or effective immune response. Dose-sparing means that a lower amount of antigen is needed to raise a desired or effective immune response, that would otherwise be the case. Dose- and dosage-sparing are meant to overcome technical problems associated with the development of a vaccine against a pre-pandemic or pandemic influenza virus, such as the weak immune response that avian or swine viral HA elicits in humans. During development of prior pandemic flu vaccines, a large multicenter trial found that two injections of 90 μg H5 given 28 days apart provided protection in only 54% of humans (Treanor et al., New England Journal of Medicine 354:1343-1351, 2006). It is estimated that the world is currently capable of producing only 70 million doses of pandemic flu vaccine administered as two injections of 90 μg within a desired timeframe (Poland, G. A., New England Journal of Medicine, 354:1411-1413, 2006). Preferred vaccine formulations reduce the amount of protein required for vaccination, i.e., to achieve seroconversion, through either dose-and or dosage-sparing.

Any of the assays described herein can be used to verify dose- and/or dosage-sparing. An exemplary assay to verify dose and dosage-sparing is the hemagglutination inhibition (HAI) assay that measures serum antibody response to vaccination. The FDA has established guidelines for pandemic influenza vaccine evaluation that state that an HAI titer greater than or equal to 40 is a suitable immunological parameter for predicting protection from natural infection (see Food and Drug Administration 2007 Guidance for industry: clinical data needed to support the licensure of seasonal inactivated influenza vaccines). As used herein, a person who has an HAI titer greater than or equal to 40 will be considered as a person who has achieved seroconversion. An exemplary dose of antigen is the amount of antigen in a vaccine formulation effective to achieve an HAI titer of about 40 in about 50% of individuals vaccinated once, i.e., one time only, with the antigen formulation. Another exemplary dose of antigen is the amount of antigen in a vaccine formulation effective to achieve an HAI titer of about 40 in about 70% of individuals vaccinated once with the antigen formulation. Another exemplary dose of antigen is the amount of antigen in a vaccine formulation effective to achieve an HAI titer of about 40 in about 80% of individuals vaccinated once with the antigen formulation.

The pharmaceutical compositions, vaccine compositions, and kits described herein may be administered to individuals to prevent or protect against influenza infections or to treat disease following infection. The administration may be performed at a pre-pandemic stage or during a pandemic.

Subjects to receive the compositions include high-risk individuals, such as those in close contact or likely to be in contact with sick or dead animals, e.g., birds (for H5N1 virus), selected populations in order to “prime” against a pandemic, children, elderly, pregnant women, people with certain chronic medical or immunosuppressive conditions, and ideally the world's population.

Influenza may be avoided by administration of the compositions as a single dose (e.g., injection) or as multiple doses. When multiple doses are administered, generally the second and subsequent doses are administered after an interval of time. Often administration of the initial dose is called “priming” the immune response, and administration of subsequent doses are called “boosting” the immune response. Typically, the time between the first and second administration is at least 2 weeks, although shorter or longer time periods may be used. Additional doses may be administered at least 2-4 weeks following the earlier administration, and in some cases, may be administered long (e.g., 1 year) after the earlier dose. Administration of dose(s) following influenza infection serves to treat disease.

Because HA sequences of pandemic viruses can drift and because at a pre-pandemic stage it isn't known which of the potential viruses will become pandemic, it is desirable to administer vaccines that provide broad coverage against related viruses. As shown herein, antibodies to related viruses were obtained from administration of one rHA protein. Another way to obtain broad coverage may be to prime with one rHA and boost with a different rHA.

The compositions may be administered along with other antiviral agents. Antiviral agents are medicines, drugs, herbs, etc. that act directly on viruses to stop them from multiplying. Some well-known antiviral agents include Tamiflu® (oseltamivir), amantadine (Symmetrel®), rimantadine (Flumadine®), zanamivir (Relenza®), peramivir, laninamivir. Other neuraminidase inhibitors and M2 inhibitors may also be available. Chinese herbs may also be administered along with the compositions. Other agents include those that treat symptoms, such as cough syrup, aspirin, NSAIDs such as ibuprofen may also be provided.

The following examples are offered by way of illustration, and not by way of limitation.

### EXAMPLES

### Example 1

### Efficacy of a Single Injection RH5/GLA-SE Flu Vaccine in Mice

This example demonstrates that a single vaccination with recombinant influenza H5 (rH5) protein can effectively prime a protective anti-viral immune response in mice challenged with a high titer of H5N1 virus. Balb/c mice (5/group) were injected intramuscularly (IM) once with increasing amounts (0, 50, 150, 450, 900, or 2700 ng) of rH5 protein (derived from H5N1 Viet Nam 1203; available from Protein Sciences, Meriden, Conn.) alone or rH5 protein formulated with GLA-SE adjuvant (20 μg of GLA in 2% SE). Mice were challenged 14 days later by intranasal administration of H5N1 Viet Nam 1203 (1000×LD₅₀). Mice were monitored daily for weight loss and euthanized if loss of weight was greater than 20-30%. Vaccination with rH5 protein alone did not provide protective immunity as all animals injected with rH5 in the absence of adjuvant either died spontaneously following viral challenge (18/25 animals) or displayed significant morbidity and were euthanized (7/25 animals). However, all mice vaccinated with rH5+GLA-SE adjuvant survived (25/25 animals), even at the lowest dose of rH5 protein administered. These results indicate that a single injection of a recombinant subunit influenza vaccine can confer protective immunity to mice when formulated with GLA-SE adjuvant. This protective immunity was observed despite a 50-fold reduction in antigen dose.

The benefits of adding GLA adjuvant to rH5 vaccine were further explored by examining weight loss in vaccinated animals following viral challenge. Balb/c mice (5/group) were injected once with increasing amounts of rH5 protein alone (0, 50, 150, 450, 900, or 2700 ng) or rH5 protein formulated with 20 μg GLA-SE adjuvant or SE emulsion alone (100 μL of a 2% solution). Mice were challenged 14 days later with H5N1 Viet Nam 1203 (1000×LD₅₀) and weight was measured 14 days following challenge. Mice vaccinated with rH5 protein alone lost considerable weight following viral challenge and died before demonstrating any recovery, even at the highest dose of vaccine administered. In contrast, all animals vaccinated with rH5 protein formulated with GLA-SE adjuvant survived viral challenge and were able to re-establish body weight. Mice vaccinated with rH5 protein formulated with SE emulsion alone also recovered from viral challenge and gained weight.

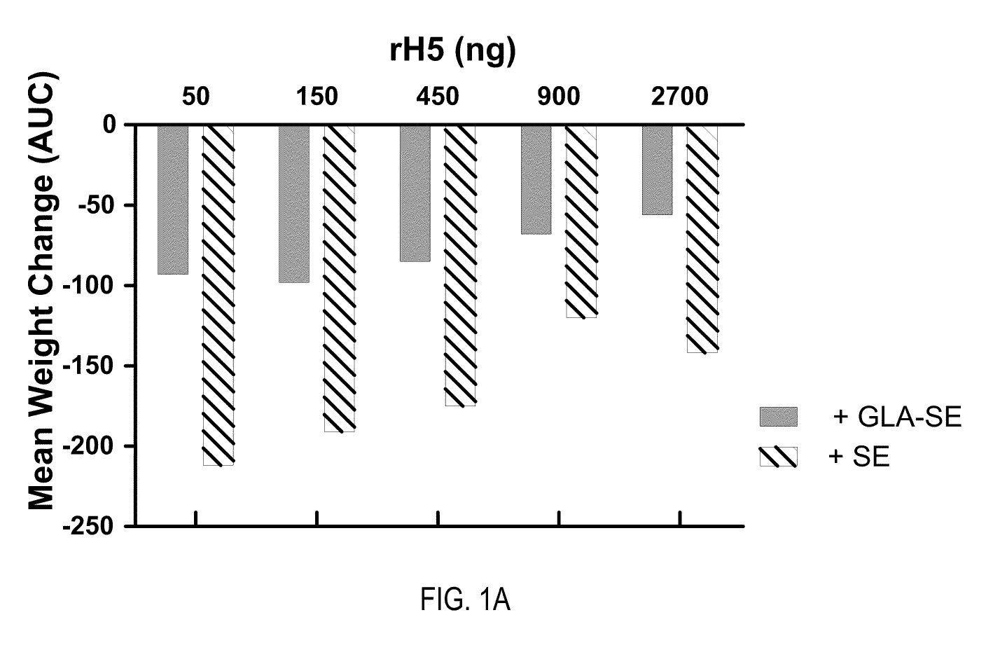

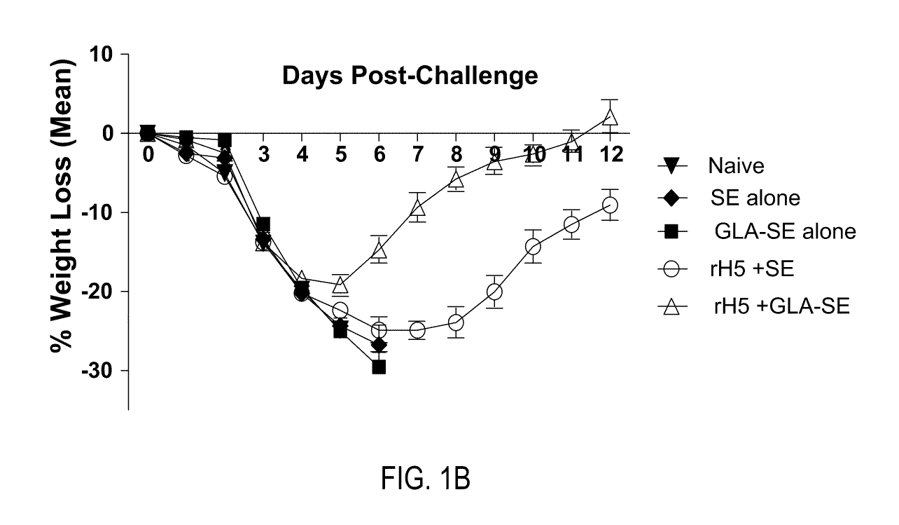

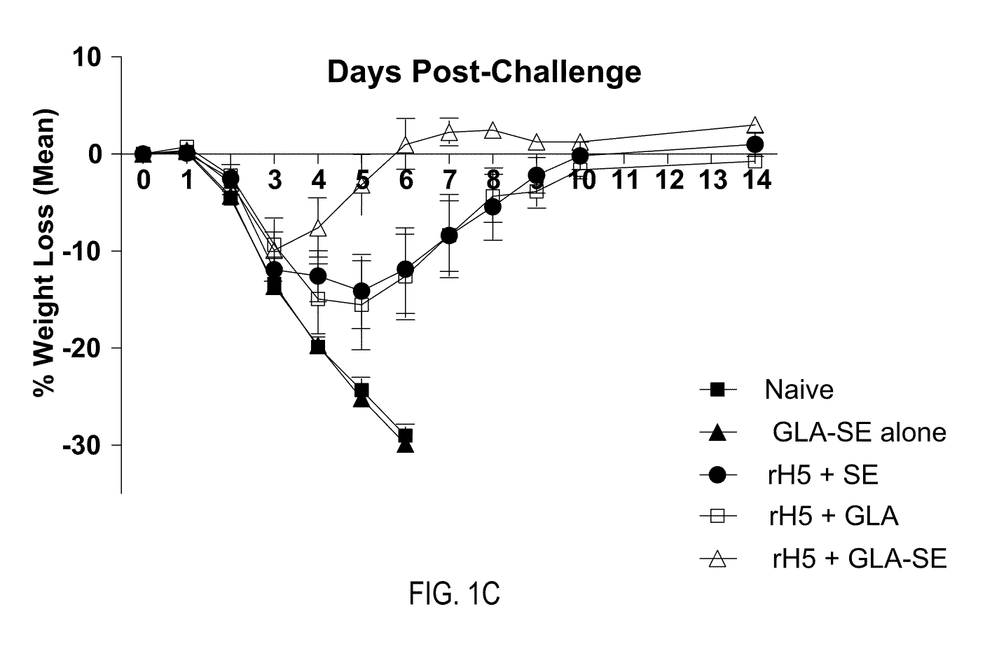

To quantitate the difference in recovery between these two groups, the mean percentage weight change over the 14 day test period for all groups was calculated by measuring the area under the curves representing daily weight change over the two week test period. A bar graph depicting these values is shown in FIG. 1A, which indicates that animals receiving rH5 formulated with GLA-SE lose considerably less weight than those receiving rH5 formulated in SE emulsion alone. These results demonstrate that rH5 formulated with GLA adjuvant induces superior protection at all antigen doses. Thus, addition of GLA adjuvant to rH5 protein yields a greatly improved vaccine that establishes protective immunity when administered as a single, low dose injection in mice. These results suggest that formulation with GLA adjuvant offers a potential solution to some of the challenges associated with developing a recombinant protein based vaccine for pandemic flu, namely augmentation of antigen immunogenicity such that only a single vaccine injection is required to establish protective immunity. Dosage-sparing of vaccine is an urgent priority for public health authorities in preparing for potential flu pandemic.

The improved properties of the rH5 vaccine comprised of GLA adjuvant were further studied by measuring the kinetics weight change in vaccinated animals following viral challenge. Balb/c mice (5/group) were injected once with 50 ng rH5 protein formulated in GLA-SE adjuvant or SE emulsion alone and then challenged 14 days later with H5N1 Viet Nam 1203 (1000×LD₅₀), as described above. As controls, mice were vaccinated with GLA-SE adjuvant or SE emulsion in the absence of rH5 protein. Mice were weighed each day following viral challenge and percent weight loss was determined relative to the weight of animals prior to challenge. In this challenge model, unimmunized, naïve, mice do not recover from viral challenge, as evidenced by rapid weight loss observed followed by death. As shown in FIG. 1B, all mice that survive viral challenge showed symptoms of infection as their weights initially declined at rates identical to that observed in the unimmunized, control group. However, mice immunized with rH5 formulated with GLA-SE recovered from viral challenge as shown by weight gain that returns to pre-vaccination levels 10-12 days following viral challenge. Mice immunized with rH5 formulated with SE emulsion alone also recovered, however, their rate of recovery was significantly delayed relative to that observed in animals vaccinated with rH5 formulated with GLA-SE adjuvant. This protection was dependent on rH5 protein, as mice immunized with GLA-SE adjuvant or SE emulsion alone responded to viral challenge in a manner similar to that of naïve mice. Importantly, these data indicate that the improved efficacy of the single injection, low dose rH5 vaccine is dependent upon the combined activities of rH5 protein and GLA adjuvant.

To further define the components necessary for the improved efficacy of the rH5 vaccine formulated in GLA adjuvant, the kinetics of weight change were measured in animals vaccinated with GLA-SE adjuvant alone, rH5 protein+SE alone, rH5+GLA alone, or rH5+GLA-SE. As shown in FIG. 1C, unimmunized control mice and mice immunized with GLA-SE in the absence of rH5 protein lost weight dramatically and died following viral challenge, as previously observed. In contrast, mice immunized with the combination of rH5 and GLA-SE recovered from viral challenge and re-established full weight. Mice immunized with rH5 formulated with either SE alone or GLA alone also recovered, however, the rate of recovery observed in these two groups was considerably delayed relative to that of mice receiving the rH5+GLA-SE vaccine. These data indicate that the combination of rH5 and GLA adjuvant in SE emulsion displays superior properties relative to those of any individual component.

### Example 2

### RH5/GLA-SE Flu Vaccine Confers Heterolgous Immunity in Mice When Administered as a Single Injection

In this example, the protective efficacy of recombinant vaccine formulated with GLA adjuvant against heterologous viral challenge was demonstrated. For these experiments, mice were immunized with a single injection of the rH5 protein isolated from H5N1 Indonesia (clade 2.3), followed by challenge with the H5N1VN virus, as described above. As a positive control, mice were vaccinated with the homologous rH5 protein from H5N1Vietnam, while as a negative control, mice were vaccinated with an unrelated HSV-2 viral protein (rG013). As shown in Table 1, mice vaccinated with the HSV-2 protein all died, irrespective of the protein-adjuvant formulation. All mice vaccinated with 50 ng of homologous rH5VN protein alone died, while all mice vaccinated with rH5VN formulated with GLA-SE adjuvant survived, consistent with previous findings. Importantly, all mice receiving either 50 ng or 200 ng of the heterologous rH5 Indo protein formulated with GLA-SE also survived, demonstrating that GLA-SE effectively broadens cross-clade protective immunity. Interestingly, at the lowest dose of rH5 Indo (50 ng) administered, formulation of protein with SE alone failed to protect mice from viral challenge (no mice surviving), while formulation with GLA in the absence of SE emulsion showed protection in 40% of mice (2/5).

TABLE 1

| antigen | (ng) | rH5 alone | rH5 + SE | rH5 + GLA | rH5 + GLA-SE |
|---|---|---|---|---|---|
| rH5 VN | 50 | 0/5 | 5/5 | 5/5 | 5/5 |
| rH5 Indo | 50 | 0/5 | 0/5 | 1/5 | 5/5 |
| rH5 Indo | 200 | 0/5 | 2/5 | 2/5 | 5/5 |
| rG103 | 200 | 0/5 | 0/5 | 0/5 | 0/5 |

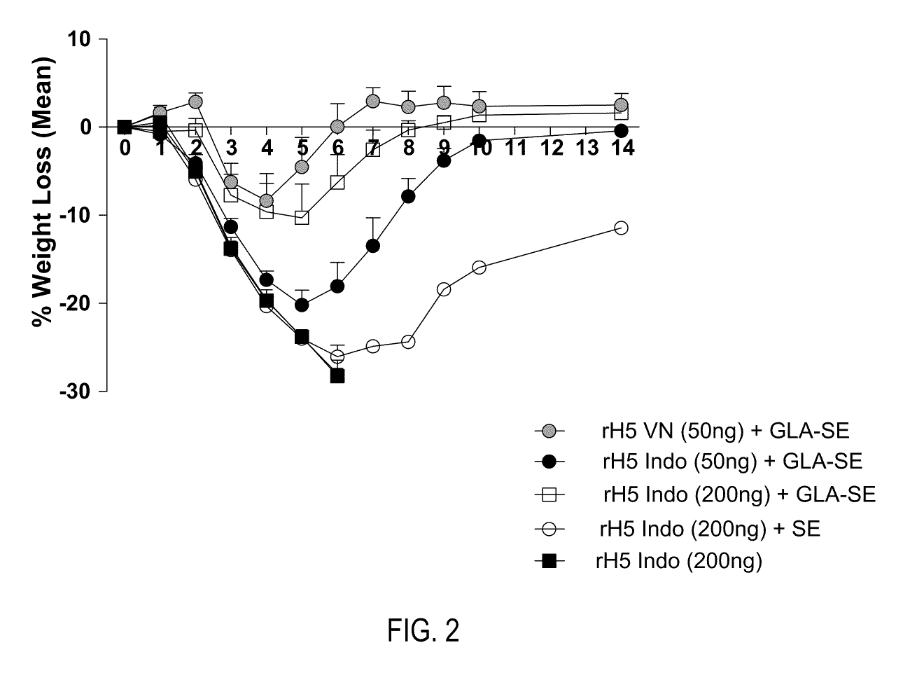

The improved efficacy the rH5 Indo vaccine when formulated with GLA adjuvant was also observed when recovery from weight loss was monitored following viral challenge, as shown in FIG. 2. As observed in previous experiments, mice vaccinated with rH5 protein alone did not recover from viral challenge, while mice receiving rH5 formulated with GLA-SE adjuvant displayed rapid recovery and gained weight back to their pre-challenge levels. Mice vaccinated with heterologous rH5 Indo protein formulated in GLA-SE adjuvant also recovered quickly, with kinetics that depended on the dose of recombinant protein. Thus, GLA-SE improves the efficacy of both homologous and heterologous recombinant flu vaccines. Establishment of cross-clade protective immunity with a dose and dosage sparing vaccine is an especially advantageous property of a candidate pandemic flu vaccine.

### Example 3

### GLA-SE Accelerates Establishment of Antigen-Specific Immunity in Mice

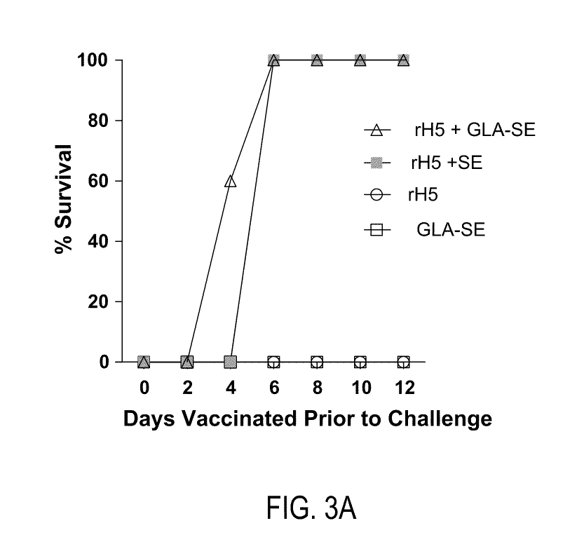

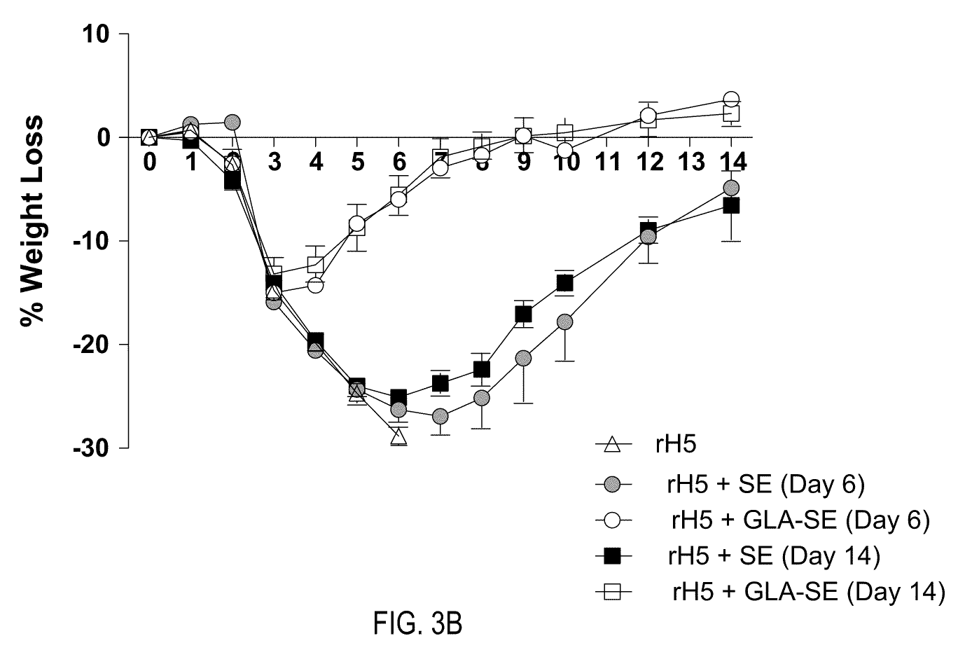

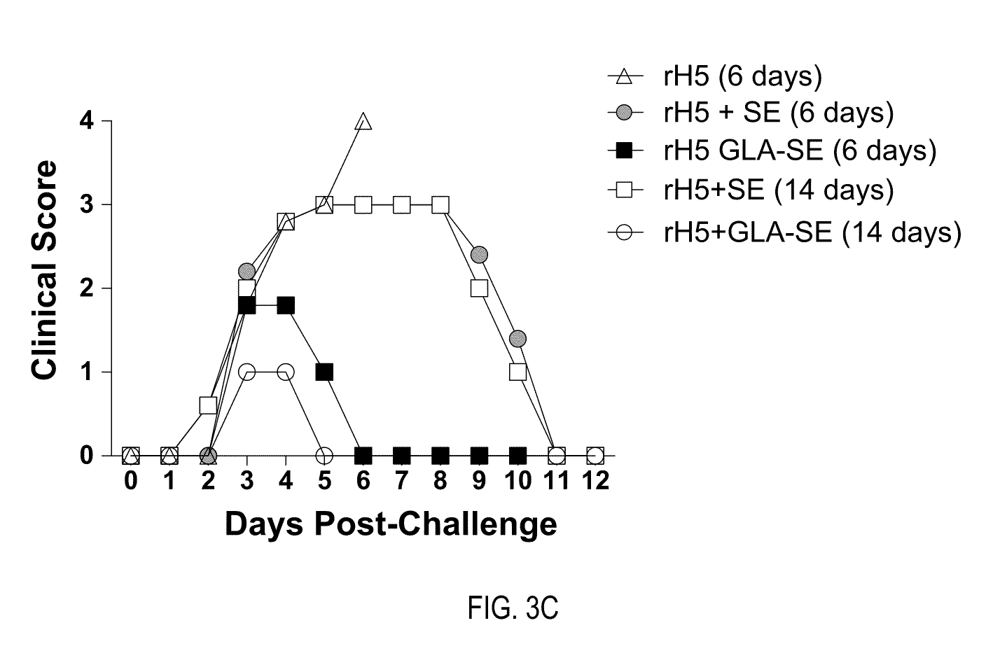

This example demonstrates the temporal requirements for establishing immunity in the mouse protection model by challenging mice with influenza virus at successive days following immunization. Mice were vaccinated with a single injection of a low dose of rH5 protein formulated in SE alone or GLA-SE adjuvant, as previously described. As controls, mice were vaccinated with rH5 protein or GLA-SE alone. Mice were challenged at various days (0, 2, 4, 6, 8, 10, or 12 days) following vaccination and percent survival was determined 14 days later. As shown in FIG. 3A, rH5 protein formulated with GLA adjuvant established protective immunity within 4-6 days post-vaccination. As expected, this effect was dependent upon both recombinant protein and GLA-SE, as mice receiving vaccines lacking either of these components all died. Mice receiving rH5 formulated in SE alone also demonstrated protection from viral challenge, although acquisition of protective immunity in this group was delayed by over a day relative to that observed in the rH5+GLA-SE group.

The kinetics of weight loss were measured in mice challenged with virus at either day 6 or day 14 following vaccination, as previously described. As shown in FIG. 3B, when mice were challenged six days after immunization, the rH5+SE emulsion alone group lost significantly more weight as compared to the group receiving rH5+GLA-SE adjuvant. This difference observed between groups did not diminish upon delay of challenge to day 14 following vaccination (see FIG. 3B). These data indicate that not only does vaccination with rH5 formulated in GLA adjuvant lead to less weight loss following viral challenge, it also enables animals to recover significantly more quickly. A similar trend in general health was observed using a clinical scoring method (see FIG. 3C). Mice treated with rH5+GLA-SE appeared less sick and recovered faster than the rH5+SE alone group regardless of the time of vaccination. Thus, GLA-SE adjuvant accelerates establishment of antigen-specific immunity relative to the SE emulsion alone. The ability of a dose- and dosage-sparing recombinant vaccine to rapidly induce protective immunity is a highly desirable property of a pandemic flu vaccine, which should be effective against unexpectedly broad and rapid viral transmission.

### Example 4

### Vaccination with RH5 Protein Formulated in GLA-SE Establishes Highly Durable Protective Immunity in Mice

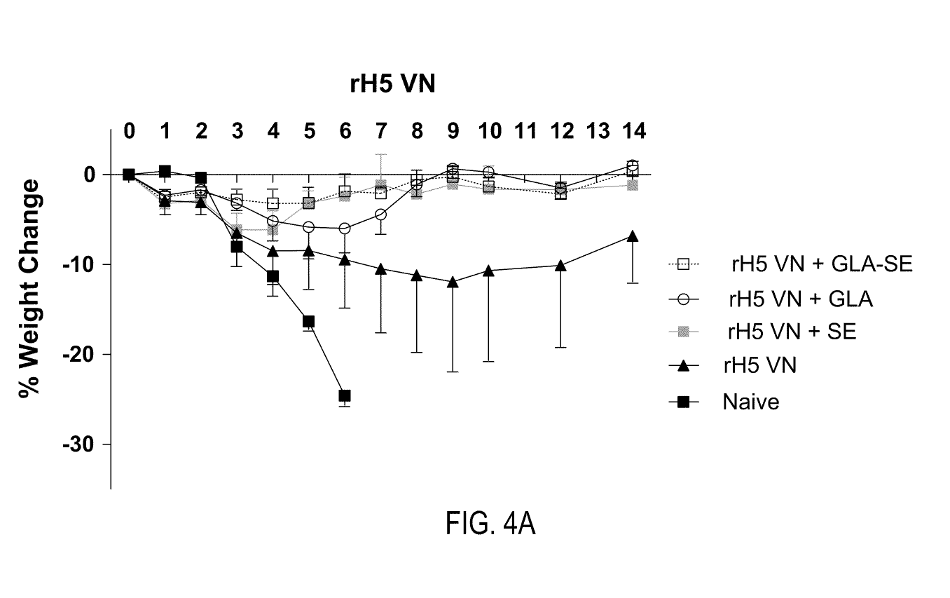

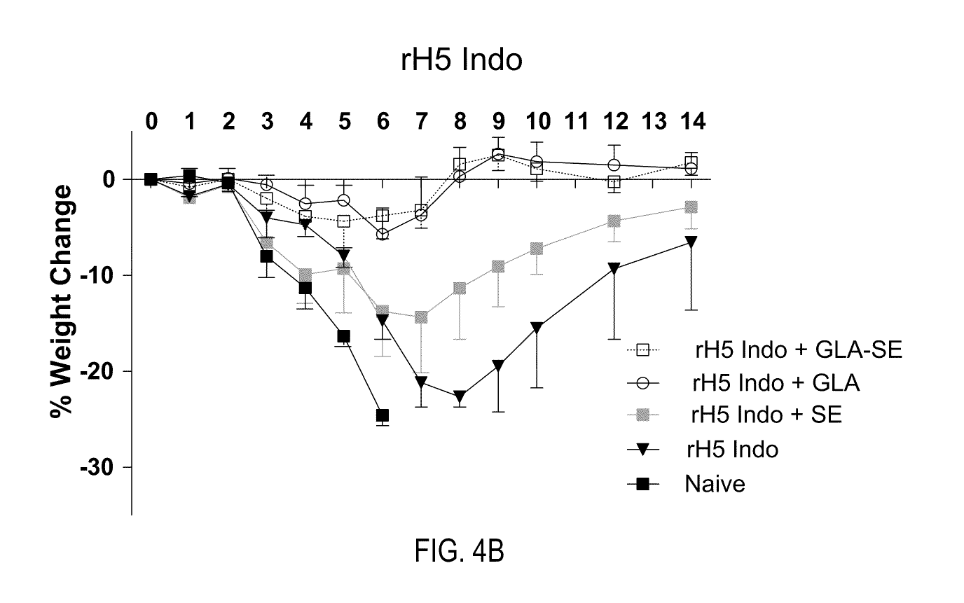

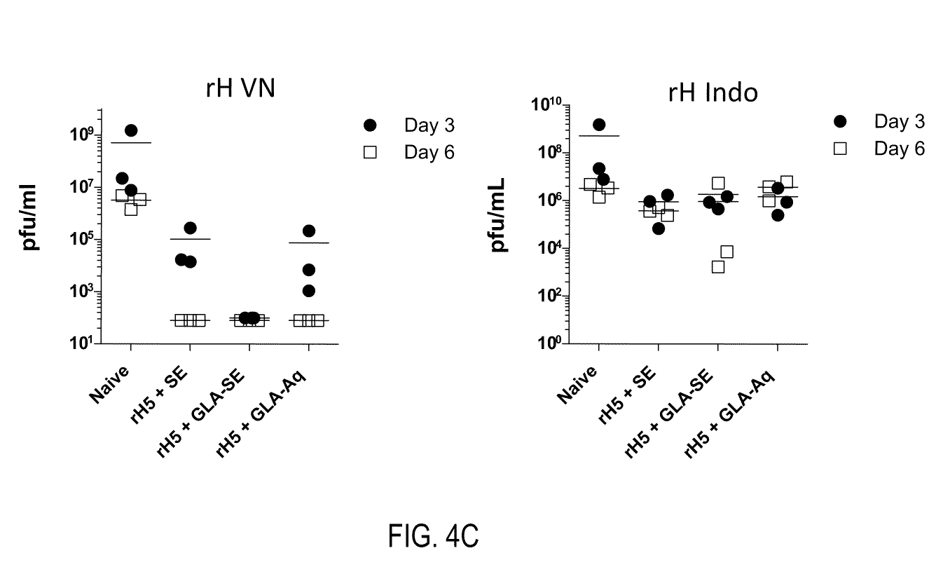

In this example, the durability of adjuvant-dependent protection was evaluated by immunizing mice once with a low dose of homologous (rH5VN) or heterologous (rH5lndo) antigen followed by viral challenge 46 days later with H5N1VN. As shown in Table 2, all mice vaccinated with recombinant H5 antigen formulated with GLA adjuvant survived viral challenge at 46 day following vaccination, regardless of whether vaccination was with the homologous or heterologous rH5 protein. Furthermore, as shown in FIGS. 4A and B, animals in this group recovered from viral challenge very quickly and lost very little body weight. FIG. 4C indicates that viral load in these groups was also reduced. Importantly, these results indicate that a vaccine composed of the combination of low dose rH5 protein and GLA adjuvant confers an effective and durable cross-clade protection to influenza virus in mice.

TABLE 2

| Antigen | (ng) | rH5 | rH5 + SE | rH5 + GLA | rH5 + GLA-SE |
|---|---|---|---|---|---|
| rH5 VN | 50 | 3/5 | 3/5 | 5/5 | 5/5 |
| rH5 Indo | 50 | 2/5 | 4/5 | 3/5 | 5/5 |

### Example 5

### Efficacy of a Single injection of RH5/GLA-SE Flu Vaccine in Ferrets

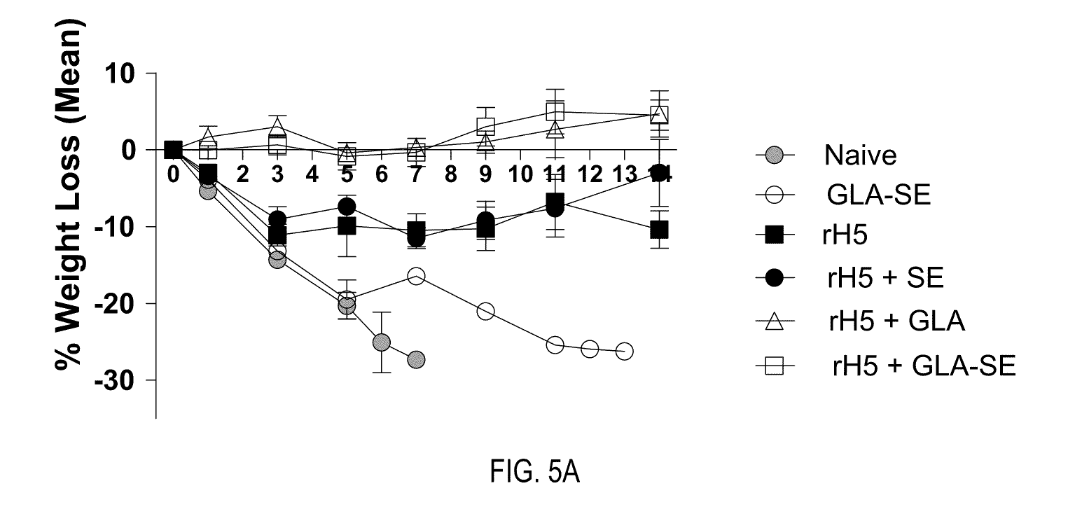

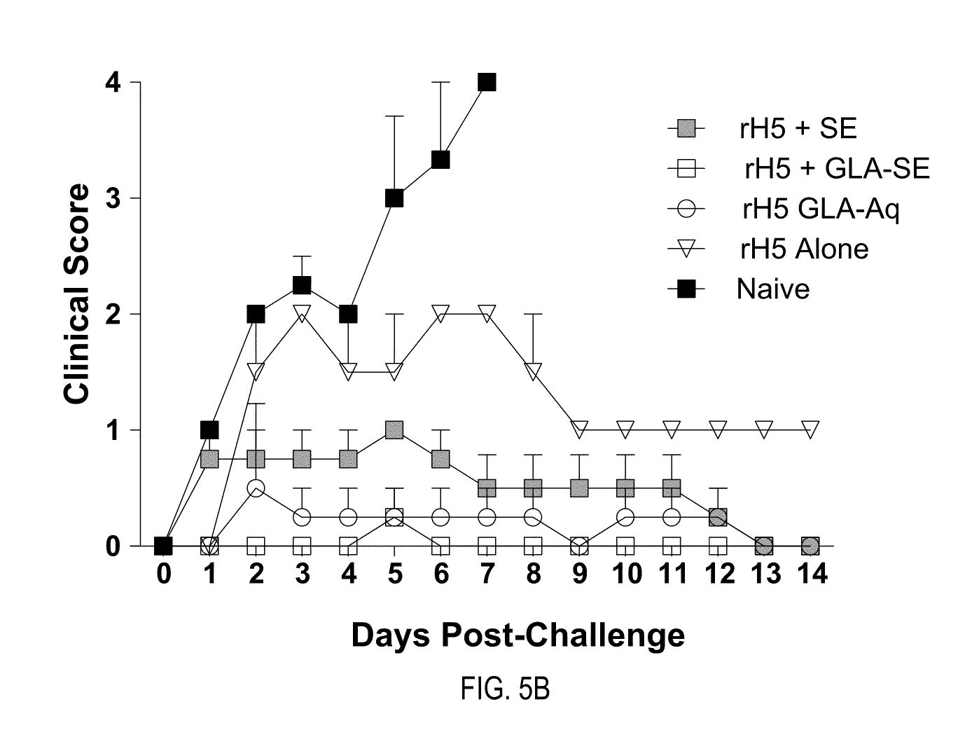

The experiments described in this example address whether protective immunity can be established by a single injection of a low dose rH5 vaccine in ferrets, which are a suitable preclinical host for flu vaccine development. Male Fitch ferrets, 6-12 months of age (Triple F Farms, Sayre, Pa.) were used for all experiments. Prior to inoculation, all animals were confirmed to be serologically negative for circulating seasonal influenza (influenza A H1N1, H3N2, and Influenza B) by haemagglutinin inhibition (HI) assay. For all experiments, ferrets were housed in cages contained within a Duo-Flo Bioclean mobile clean room (Lab Products, Seaford, Del.). Baseline serum, temperature and weight data were taken daily for approximately 3 days prior to infection. Temperatures were measured using a subcutaneous implantable temperature transponder (BioMedic Data Systems, Seaford Del.). Ferrets (four per group) were vaccinated once with 0.5 pg rH5VN, either alone or with adjuvant, and then challenged on day 28 post-vaccination with H5N1VN. Ferrets were inoculated intra-nasally with 7.5×105 PFU of A/VN/1203/05 (H5N1) virus in a total volume of 1 mL. Nasal washes were collected from all ferrets every 24 hours beginning 1 day following infection and continuing for 7 days. Any animal losing >25% of their day 0 body weight, exhibiting neurological symptoms, or determined to be in a moribund state was humanely euthanized. As shown in Table 3, all animals injected with rH5 plus SE, GLA, or GLA-SE survived viral challenge, while vaccination with rH5 protein lacking adjuvant or GLA-SE adjuvant lacking flu antigen failed to protect all animals from virus. When the kinetics of weight loss following viral challenge were measured, it was observed that animals vaccinated with rH5 vaccine formulated with GLA adjuvant lost very little weight, in contrast to animals vaccinated with rH5 alone or rH5 formulated in SE emulsion (see FIG. 5A). In this animal model, optimal efficacy of the rH5+GLA vaccine in ferrets did not appear to require the SE emulsion. This trend was recapitulated when clinical score of each group was determined, as shown in FIG. 5B. Animals receiving rH5 formulated in GLA adjuvant appeared normal based on clinical observation, in contrast to animals receiving rH5 alone. Collectively, these results demonstrate that a single injection of a low dose rH5 vaccine containing GLA adjuvant effectively protects ferrets against H5N1 infection. Thus, the ability of GLA adjuvant to substantially improve the efficacy of a single injection, low dose recombinant H5 vaccine has been demonstrated in two different animal models of protective immunity.

TABLE 3

| antigen | (ng) | naive | GLA-SE | rH5 | rH5 + SE | rH5 + GLA | rH5 + GLA-SE |
|---|---|---|---|---|---|---|---|
| rH5 VN | 50 | 0/4 | 1/4 | 2/4 | 4/4 | 4/4 | 4/4 |

From the foregoing it will be appreciated that, although specific embodiments have been described herein for purposes of illustration, various modifications may be made without deviating from the spirit and scope of the invention. Accordingly, the invention is not limited except as by the appended claims.

We claim:

1. A pharmaceutical composition comprising a recombinant hemagglutinin (rHA) from a pre-pandemic or pandemic influenza virus and an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (−O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons, and wherein the composition is dosage sparing.

2. The composition of claim 1, wherein the rHA is present at an amount that is dose-sparing.

3. The composition of claim 2, wherein the rHA is present at a concentration that does not provide protective immunity in the absence of the adjuvant.

4. The composition of claim 1, wherein the rHA is from a pathogenic strain of avian influenza.

5. The composition of claim 4, wherein the rHA is from a pathogenic strain of H5N1 influenza.

6. The composition of claim 5, wherein the rHA is from clade 1 or clade 2.

7. The composition of claim 1, wherein the rHA is from a pandemic swine flu virus strain.

8. The composition of claim 7, wherein the rHA is from a pandemic H1 N1 strain.

9. The composition of claim 1, wherein the composition comprises a single recombinant protein.

10. The composition of claim 1, wherein the amount of rHA per dose is in the range of about 15 to about 0.1 μg.

11. The composition of claim 1, wherein the rHA is expressed in insect or mammalian cells

12. The composition of claim 1, wherein the rHA is expressed as a fusion protein.

13. The composition of claim 1, wherein the adjuvant is GLA.

14. The composition of claim 1, wherein the composition is aqueous and oil-free or comprises less than about 1% v/v oil.

15. The composition of claim 1, wherein the adjuvant is formulated in combination with a surfactant and water, but no oil, prior to being combined with rHA to form the composition.

16. The composition of claim 1, wherein the adjuvant is formulated in a liposome.

17. A method of immunizing a subject against a pre-pandemic or pandemic influenza virus comprising administering a pharmaceutical composition comprising a recombinant hemagglutinin (rHA) from a pre-pandemic or pandemic influenza virus and an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons, and wherein the composition is dosage sparing.

18. The method of claim 17, wherein the adjuvant is GLA and the rH5 is from a pathogenic strain of H5N1 influenza.

19. The method of claim 17, wherein the pharmaceutical composition is administered by a single injection.

20. The method of claim 17, wherein the pharmaceutical composition comprises a single rHA but provides protection against more than one strain of H5N1 influenza.

21. The method of claim 17, wherein the rH5 is from an influenza virus of a different clade than the pre-pandemic or pandemic influenza virus.

22. A method of immunizing a subject in need thereof against a pre-pandemic or pandemic influenza virus, comprising administering a single injection of a pharmaceutical composition comprising
(a) a recombinant hemagglutinin (rHA) from a pre-pandemic or pandemic influenza virus and
(b) an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons, where the administration achieves seroconversion after the single injection.

23. The method of claim 22 wherein the composition does not include an emulsion.

24. The method of claim 22 wherein the adjuvant is GLA.
